SEQUENCE LISTING (70 sequences)

SEQ ID NO: 1 - protein, 638 aa, synthetic construct
  REGION 1..638: note = Synthetic: RSLV-145 (DNase-NLG linker-RNase-Fc) amino acid sequence
LKIAAFNIQTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTAKPTHCAYDRIVVAGMLLRGAVVPDSALPFNFQAAYGLSDQLAQAISDHYPVEVMLKVDGASSPVNVSSPSVQDIKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 638 aa, synthetic construct
  REGION 1..638: note = Synthetic: RSLV-146 (Fc-linker-RNase-NLG linker-DNase) amino acid sequence
DKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTVDGASSPVNVSSPSVQDILKIAAFNIQTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTAKPTHCAYDRIVVAGMLLRGAVVPDSALPFNFQAAYGLSDQLAQAISDHYPVEVMLK

SEQ ID NO: 3 - protein, 492 aa, synthetic construct
  REGION 1..492: note = Synthetic: RSLV-147 DNase chain (Dnase-FcA) amino acid sequence
LKIAAFNIQTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTAKPTHCAYDRIVVAGMLLRGAVVPDSALPFNFQAAYGLSDQLAQAISDHYPVEVMLKEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 4 - protein, 360 aa, synthetic construct
  REGION 1..360: note = Synthetic: RSLV-147 RNase chain (RNase-FcB) amino acid sequence
KESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 5 - protein, 638 aa, synthetic construct
  REGION 1..638: note = Synthetic: RSLV-148 (DNase-FcA-NLG linker-RNase) amino acid sequence
LKIAAFNIQTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTAKPTHCAYDRIVVAGMLLRGAVVPDSALPFNFQAAYGLSDQLAQAISDHYPVEVMLKEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGKVDGASSPVNVSSPSVQDIKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 6 - protein, 227 aa, synthetic construct
  REGION 1..227: note = Synthetic: RSLV-148 FcB chain amino acid sequence
DKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 7 - protein, 492 aa, synthetic construct; identical to SEQ ID NO: 3
  REGION 1..492: note = Synthetic: RSLV-149 DNase chain (DNase-Fc) amino acid sequence

SEQ ID NO: 8 - protein, 373 aa, synthetic construct
  REGION 1..373: note = Synthetic: RSLV-149 RNase chain (Fc-NLG-linker-RNase) amino acid sequence
DKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGKVDGASSPVNVSSPSVQDIKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 9 - protein, 510 aa, synthetic construct
  REGION 1..510: note = Synthetic: RSLV-150 DNase chain (FcA-NLG linker-DNase) amino acid sequence
EPKSCDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGKVDGASSPVNVSSPSVQDILKIAAFNIQTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTAKPTHCAYDRIVVAGMLLRGAVVPDSALPFNFQAAYGLSDQLAQAISDHYPVEVMLK

SEQ ID NO: 10 - protein, 378 aa, synthetic construct
  REGION 1..378: note = Synthetic: RSLV-150 RNase chain (FcB-NLG linker-RNase) amino acid sequence
EPKSCDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGKVDGASSPVNVSSPSVQDIKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 11 - protein, 505 aa, synthetic construct
  REGION 1..505: note = Synthetic: RSLV-151 DNase chain (Fc-NLG linker-DNase) amino acid sequence
DKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGKVDGASSPVNVSSPSVQDILKIAAFNIQTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTAKPTHCAYDRIVVAGMLLRGAVVPDSALPFNFQAAYGLSDQLAQAISDHYPVEVMLK

SEQ ID NO: 12 - protein, 373 aa, synthetic construct; identical to SEQ ID NO: 8
  REGION 1..373: note = Synthetic: RSLV-151 RNase chain (Fc-NLG linker-RNase) amino acid sequence

SEQ ID NO: 13 - protein, 638 aa, synthetic construct
  REGION 1..638: note = Synthetic: RSLV-152 (RNase-Fc-NLG linker-DNase) amino acid sequence
KESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGKVDGASSPVNVSSPSVQDILKIAAFNIQTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTAKPTHCAYDRIVVAGMLLRGAVVPDSALPFNFQAAYGLSDQLAQAISDHYPVEVMLK

SEQ ID NO: 14 - protein, 227 aa, synthetic construct; identical to SEQ ID NO: 6
  REGION 1..227: note = Synthetic: RSLV-152 Fc domain chain amino acid sequence

SEQ ID NO: 15 - protein, 505 aa, synthetic construct; identical to SEQ ID NO: 11
  REGION 1..505: note = Synthetic: RSLV-153 DNase chain (Fc-NLG linker-DNase) amino acid sequence

SEQ ID NO: 16 - protein, 360 aa, synthetic construct; identical to SEQ ID NO: 4
  REGION 1..360: note = Synthetic: RSLV-153 RNase chain (RNase-Fc) amino acid sequence

SEQ ID NO: 17 - protein, 621 aa, synthetic construct
  REGION 1..621: note = Synthetic: RSLV-154 (RNase-Fc-NLG linker-DNase) amino acid sequence
KESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPGKVDGASSPVNVSSPSVQDILKIAAFNIQTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTAKPTHCAYDRIVVAGMLLRGAVVPDSALPFNFQAAYGLSDQLAQAISDHYPVEVMLK

SEQ ID NO: 18 - protein, 505 aa, synthetic construct
  REGION 1..505: note = Synthetic: Fc-NLG-linker-DNAse (control construct)
DKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 19 - protein, 492 aa, synthetic construct
  REGION 1..492: note = Synthetic: DNASe-Fc (control construct)
LKIAAFNIQTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTAKPTHCAYDRIVVAGMLLRGAVVPDSALPFNFQAAYGLSDQLAQAISDHYPVEVMLKEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 260 aa, Homo sapiens
  REGION 1..260: note = misc_feature - Mature wild type Human DNase1
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 21 - protein, 260 aa, Homo sapiens
  REGION 1..260: note = misc_feature - Mature human DNase1 A114F
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 22 - protein, 260 aa, Homo sapiens
  REGION 1..260: note = misc_feature - Mature human DNase1 G105R
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 23 - protein, 282 aa, Homo sapiens [sequence omitted]

SEQ ID NO: 24 - protein, 260 aa, Homo sapiens
  REGION 1..260: note = misc_feature - Mature human DNase1 N18S/N106S/A114F
LKIAAFNIQTFGETKMSSATLVSYIVQILSRYDIALVQEVRDSHLTAVGKLLDNLNQDAPDTYHYVVSEPLGRNSYKERYLFVYRPDQVSAVDSYYYDDGCEPCGSDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 25 - protein, 260 aa, Homo sapiens
  REGION 1..260: note = misc_feature - Mature human DNase1 E13R/N74K/A114F/ T205K
LKIAAFNIQTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTAKPTHCAYDRIVVAGMLLRGAVVPDSALPFNFQAAYGLSDQLAQAISDHYPVEVMLK

SEQ ID NO: 26 - protein, 260 aa, Homo sapiens
  REGION 1..260: note = misc_feature - Mature human DNase1 E13R/N74K/A114F/ T205K/N18S/N106S
LKIAAFNIQTFGRTKMSSATLVSYIVQILSRYDIALVQEVRDSHLTAVGKLLDNLNQDAPDTYHYVVSEPLGRKSYKERYLFVYRPDQVSAVDSYYYDDGCEPCGSDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTAKPTHCAYDRIVVAGMLLRGAVVPDSALPFNFQAAYGLSDQLAQAISDHYPVEVMLK

SEQ ID NO: 27 - protein, 128 aa, Homo sapiens
  REGION 1..128: note = misc_feature - Mature human RNase1
KESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 28 - protein, 128 aa, Homo sapiens
  REGION 1..128: note = misc_feature - Mature human RNase1 N34S/N76S/N88S
KESRAKKFQRQHMDSDSSPSSSSTYCNQMMRRRSMTQGRCKPVNTFVHEPLVDVQNVCFQEKVTCKNGQGNCYKSSSSMHITDCRLTSGSRYPNCAYRTSPKERHIIVACEGSPYVPVHFDASVEDST

SEQ ID NO: 29 - protein, 156 aa, Homo sapiens
  REGION 1..156: note = misc_feature - Precursor human RNase1
MALEKSLVRLLLLVLILLVLGWVQPSLGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 30 - protein, 15 aa, synthetic construct
  REGION 1..15: note = Synthetic: (Gly4Ser)3 linker
GGGGSGGGGSGGGGS

SEQ ID NO: 31 - protein, 17 aa, synthetic construct
  REGION 1..17: note = Synthetic: Gaussia luciferase signal peptide
MGVKVLFALICIAVAEA

SEQ ID NO: 32 - protein, 47 aa, synthetic construct
  REGION 1..47: note = Synthetic: Linker
LEAEAAAKEAAAKEAAAKEAAAKALEAEAAAKEAAAKEAAAKEAAAK

SEQ ID NO: 33 - protein, 7 aa, synthetic construct
  REGION 1..7: note = Synthetic: O-linked glycosylation consensus
  VARIANT 2..3: note = Xaa can be any naturally occurring amino acid
  VARIANT 6: note = Xaa is Thr or Ser
CXXGGXC

SEQ ID NO: 34 - protein, 5 aa, synthetic construct
  REGION 1..5: note = Synthetic: O-linked glycosylation consensus
  VARIANT 4: note = Xaa is Glu or Asp
NSTXA

SEQ ID NO: 35 - protein, 5 aa, synthetic construct
  REGION 1..5: note = Synthetic: O-linked glycosylation consensus
NITQS

SEQ ID NO: 36 - protein, 5 aa, synthetic construct
  REGION 1..5: note = Synthetic: O-linked glycosylation consensus
QSTQS

SEQ ID NO: 37-38 - skipped (2 entries)

SEQ ID NO: 39 - protein, 5 aa, synthetic construct
  REGION 1..5: note = Synthetic: O-linked glycosylation consensus
  VARIANT 5: note = Xaa is Lys or Arg
GGSCX

SEQ ID NO: 40 - protein, 20 aa, synthetic construct
  REGION 1..20: note = Synthetic: VK3 light chain signal peptide
METPAQLLFLLLLWLPDTTG

SEQ ID NO: 41 - protein, 18 aa, synthetic construct
  REGION 1..18: note = Synthetic: NLG linker
VDGASSPVNVSSPSVQDI

SEQ ID NO: 42 - protein, 50 aa, synthetic construct
  REGION 1..50: note = Synthetic: linker
LEAEAAAKEAAAKEAAAKEAAAKALEAEAAAKEAAAKEAAAKEAAAKALE

SEQ ID NO: 43 - protein, 4 aa, synthetic construct
  REGION 1..4: note = Synthetic: Linker
GGSG

SEQ ID NO: 44 - protein, 4 aa, synthetic construct
  REGION 1..4: note = Synthetic: Linker
GSAT

SEQ ID NO: 45 - protein, 232 aa, Homo sapiens
  REGION 1..232: note = misc_feature - Human wild-type IgG1 Fc domain
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 285 aa, Homo sapiens
  REGION 1..285: note = misc_feature - Mature human DNase1L3
MRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 47 - protein, 369 aa, Homo sapiens
  REGION 1..369: note = misc_feature - Human Trex1
MGPGARRQGRIVQGRPEMCFCPPPTPLPPLRILTLGTHTPTPCSSPGSAAGTYPTMGSQALPPGPMQTLIFFDMEATGLPFSQPKVTELCLLAVHRCALESPPTSQGPPPTVPPPPRVVDKLSLCVAPGKACSPAASEITGLSTAVLAAHGRQCFDDNLANLLLAFLRRQPQPWCLVAHNGDRYDFPLLQAELAMLGLTSALDGAFCVDSITALKALERASSPSEHGPRKSYSLGSIYTRLYGQSPPDSHTAEGDVLALLSICQWRPQALLRWVDAHARPFGTIRPMYGVTASARTKPRPSAVTTTAHLATTRNTSPSLGESRGTKDLPPVKDPGALSREGLLAPLGLLAILTLAVATLYGLSLATPGE

SEQ ID NO: 48 - protein, 360 aa, Homo sapiens
  REGION 1..360: note = misc_feature - Human DNase2 alpha (NP_001366.1)
MIPLLLAALLCVPAGALTCYGDSGQPVDWFVVYKLPALRGSGEAAQRGLQYKYLDESSGGWRDGRALINSPEGAVGRSLQPLYRSNTSQLAFLLYNDQPPQPSKAQDSSMRGHTKGVLLLDHDGGFWLVHSVPNFPPPASSAAYSWPHSACTYGQTLLCVSFPFAQFSKMGKQLTYTYPWVYNYQLEGIFAQEFPDLENVVKGHHVSQEPWNSSITLTSQAGAVFQSFAKFSKFGDDLYSGWLAAALGTNLQVQFWHKTVGILPSNCSDIWQVLNVNQIAFPGPAGPSFNSTEDHSKWCVSPKGPWTCVGDMNRNQGEEQRGGGTLCAQLPALWKAFQPLVKNYQPCNGMARKPSRAYKI

SEQ ID NO: 49 - protein, 361 aa, Homo sapiens
  REGION 1..361: note = misc_feature - human DNase2 beta
MKQKMMARLLRTSFALLFLGLFGVLGAATISCRNEEGKAVDWFTFYKLPKRQNKESGETGLEYLYLDSTTRSWRKSEQLMNDTKSVLGRTLQQLYEAYASKSNNTAYLIYNDGVPKPVNYSRKYGHTKGLLLWNRVQGFWLIHSIPQFPPIPEEGYDYPPTGRRNGQSGICITFKYNQYEAIDSQLLVCNPNVYSCSIPATFHQELIHMPQLCTRASSSEIPGRLLTTLQSAQGQKFLHFAKSDSFLDDIFAAWMAQRLKTHLLTETWQRKRQELPSNCSLPYHVYNIKAIKLSRHSYFSSYQDHAKWCISQKGTKNRWTCIGDLNRSPHQAFRSGGFICTQNWQIYQAFQGLVLYYESCK

SEQ ID NO: 50 - protein, 232 aa, synthetic construct
  REGION 1..232: note = Synthetic: Fc region N83S
EPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 232 aa, synthetic construct
  REGION 1..232: note = Synthetic: Fc region with SCC
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 232 aa, synthetic construct
  REGION 1..232: note = Synthetic: Fc region with SSS
EPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 50 aa, synthetic construct; identical to SEQ ID NO: 42
  REGION 1..50: note = Synthetic: Linker

SEQ ID NO: 54 - protein, 20 aa, synthetic construct
  REGION 1..20: note = Synthetic: VK3LP leader
METPAQLLFLLLLWLPDTTG

SEQ ID NO: 55 - protein, 232 aa, synthetic construct
  REGION 1..232: note = Synthetic: Fc region with SCC, P238S, P331S mutations
EPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 232 aa, synthetic construct
  REGION 1..232: note = Synthetic: Fc region with P238S, P331S mutations
EPKCSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 1023 aa, synthetic construct
  REGION 1..1023: note = Synthetic: RSLV-327(RNase-linker-HSA-linker-DNase E13R/N74K/A114F/ T205K)
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSLKIAAFNIQTFGRTKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTAKPTHCAYDRIVVAGMLLRGAVVPDSALPFNFQAAYGLSDQLAQAISDHYPVEVMLK

SEQ ID NO: 58 - protein, 50 aa, synthetic construct
  REGION 1..50: note = Synthetic: linker
  REPEAT 1..50: note = "GGGGS" is present at least once but can repeat up to ten or more times
GGGGSGGGGSGGGGSGGGGSGGGGSGGGGSGGGGSGGGGSGGGGSGGGGS

SEQ ID NO: 59 - protein, 50 aa, synthetic construct; identical to SEQ ID NO: 58
  REGION 1..50: note = Synthetic: linker

SEQ ID NO: 60 - protein, 51 aa, synthetic construct
  REGION 1..51: note = Synthetic: linker
  REPEAT 2..51: note = "GGGGS" is present at least once but can repeat up to ten or more times
SGGGGSGGGGSGGGGSGGGGSGGGGSGGGGSGGGGSGGGGSGGGGSGGGGS

SEQ ID NO: 61 - protein, 51 aa, synthetic construct; identical to SEQ ID NO: 60
  REGION 1..51: note = Synthetic: linker

SEQ ID NO: 62 - protein, 25 aa, synthetic construct
  REGION 1..25: note = Synthetic: linker
  REPEAT 1..25: note = "GGGGS" is present at least once but can repeat up to five times
GGGGSGGGGSGGGGSGGGGSGGGGS

SEQ ID NO: 63 - protein, 20 aa, synthetic construct
  REGION 1..20: note = Synthetic: linker
GGGGSGGGGSGGGGSGGGGS

SEQ ID NO: 64 - protein, 25 aa, synthetic construct
  REGION 1..25: note = Synthetic: linker
GGGGSGGGGSGGGGSGGGGSGGGGS

SEQ ID NO: 65 - protein, 27 aa, synthetic construct
  REGION 1..27: note = Synthetic: linker
  REPEAT 2..26: note = "EAAAK" repeats at least two times and up to five times
AEAAAKEAAAKEAAAKEAAAKEAAAKA

SEQ ID NO: 66 - protein, 10 aa, synthetic construct
  REGION 1..10: note = Synthetic: linker
  REPEAT 1..10: note = "GS" is present at least once and can repeat up to five times
GSGSGSGSGS

SEQ ID NO: 67 - protein, 20 aa, synthetic construct
  REGION 1..20: note = Synthetic: linker
  REPEAT 1..20: note = "GGSG" is present at least once and can repeat up to five times
GGSGGGSGGGSGGGSGGGSG

SEQ ID NO: 68 - protein, 30 aa, synthetic construct
  REGION 1..30: note = Synthetic: linker
  REPEAT 1..30: note = "GGSGGS" is present at least once and can repeat up to five times
GGSGGSGGSGGSGGSGGSGGSGGSGGSGGS

SEQ ID NO: 69 - skipped

SEQ ID NO: 70 - protein, 4 aa, synthetic construct
  REGION 1..4: note = Synthetic: linker
GGSG

(19) United States
(12) Patent Application Publication
(10) Pub. No.: US 2025/0002885 A1
(43) Pub. Date: Jan. 2, 2025

(54) OPTIMIZED BINUCLEASE FUSIONS AND METHODS

(71) Applicant: Resolve Therapeutics, LLC, Miami, FL (US)

(72) Inventors: James Arthur Posada, Miami, FL (US); Sanjay Patel, San Diego, CA (US); Weihong Yu, San Diego, CA (US); Chris Gabel, Seattle, WA (US)

(21) Appl. No.: 18/768,662

(22) Filed: Jul. 10, 2024

Related U.S. Application Data
(62) Division of application No. 17/560,522, filed on Dec. 23, 2021, now Pat. No. 12,077,790.
Application No. 17/560,522 is a continuation of application No. 16/313,656, filed on Dec. 27, 2018, now abandoned.
(60) Provisional application No. 62/357,756, filed on Jul. 1, 2016.

(51) Int. Cl. C12N 9/22; A61K 38/00

(52) U.S. Cl. CPC C12N 9/22; C12Y 301/21001; C12Y 301/27005; A61K 38/00; C07K 2317/72; C07K 2319/30

(57) ABSTRACT

The invention provides for optimized binuclease fusion proteins with increased pharmacokinetic properties. The optimized binuclease fusion proteins of the invention two or more nuclease domains (e.g., RNase and DNase domain) operably coupled to an Fc domain. The invention also provides methods of treating or preventing a condition associated with an abnormal immune response.

### RELATED APPLICATIONS

This application is a continuation of U.S. patent application Ser. No. 16/313,656, filed on Dec. 27, 2018, allowed, which is a 35 U.S.C. § 371 national stage filling of International Application No. PCT/US2017/040267, filed on Jun. 30, 2017, which claims the benefit of U.S. provisional Patent Application No. 62/357,756, filed Jul. 1, 2016. The entire contents of each of these applications are incorporated herein by reference in their entirety.

### STATEMENT REGARDING SEQUENCE LISTING

The application contains a Sequence Listing which has been submitted in ASCII format via EFS-Web and is hereby incorporated by reference in its entirety. Said ASCII copy, created on Dec. 22, 2021, is named “RSLV_013_C01US_Sequence-Listing.txt” and is 145,273 bytes in size.

### BACKGROUND

Accumulation of (ribo)nucleoprotein particles from dead and dying cells is known to induce an inflammatory cascade in patients with systemic lupus erythematosus (SLE) by at least two mechanisms: (i) Deposition or in situ formation of chromatin/anti-chromatin complexes causes nephritis and leads to loss of renal function; and (ii) nucleic acids complexed with autoantibodies activate innate immunity through toll-like receptor (TLR) 7, 8, and 9 as well as TLR-independent pathway(s). Release of nucleoproteins can serve as a potent antigen for autoantibodies in SLE, providing amplification of B cell and DC activation through co-engagement of antigen receptors and TLRs. Thus, there exists a need for a means to remove the nucleic acid bound to autoantibody antigens and/or attenuate immune stimulation, immune amplification, and immune complex mediated disease in subjects in need thereof, for example, with long-acting nuclease molecules that attack circulating immune complexes by digesting nucleic acids contained therein.

### SUMMARY OF THE INVENTION

The invention relates, in part, to optimized binuclease fusion proteins which are tandem binuclease fusion proteins or heterodimeric binuclease fusion proteins which are capable of binding multiple substrates with high nuclease activity. In some aspects, the tandem binuclease fusion proteins comprise one or more DNase1 and one or more RNase1 domains operably linked in tandem to one or more Fc domains. In some aspects, the heterodimeric binuclease fusion proteins comprise a single DNase1 domain and a single RNase1 domain operably linked to one or more Fc domains, such that the DNase1 and RNase1 domains are positioned at either the N- or C-terminus of the Fc domain. In some aspects, the optimized binuclease fusion proteins alleviate the problem of expressing dual-functional nuclease-Fc chains and mitigate potential steric hindrance of one or more nuclease domains. In some aspects, the heterodimeric binuclease fusion proteins comprise one or more mutations in the Fc domain(s) to maximize formation of heterodimers.

In some embodiments the optimized binuclease fusion proteins are tandem binuclease fusion proteins comprising a first nuclease domain, a second nuclease domain and an Fc region, wherein the first nuclease domain is DNase1 and the second nuclease domain is RNase1, wherein the DNase1 is operably linked with or without a linker in tandem from N- to C-terminus to the RNase1, and the RNase1 is operably linked to the N- or C-terminus of an Fc region. The tandem binuclease fusion protein exhibits enhanced pharmacokinetic activity relative to the either first or second nuclease domain alone. Such tandem binuclease fusion proteins exhibit altered, e.g., improved, serum half-life relative to either the first or second nuclease domain alone.

In some aspects the optimized binuclease fusion proteins are heterodimeric binuclease fusion proteins comprising a first nuclease domain, a second nuclease domain and an Fc region, wherein the first nuclease domain is DNase1 and the second nuclease domain is RNase1, wherein the DNase1 is operably linked with or without a linker to the N- or C-terminus of an Fc region, and the RNase1 is operably linked with or without a linker to the N- or C-terminus of an Fc region, thereby forming a heterodimer. The heterodimeric binuclease fusion protein exhibits enhanced pharmacokinetic activity relative to the either first or second nuclease domain alone. Such heterodimeric binuclease fusion proteins exhibit altered, e.g., improved, serum half-life relative to either the first or second nuclease domain alone.

In some aspect the optimized binuclease fusion proteins are those represented in FIG. 1.

In some aspects, the invention provides an optimized binuclease fusion protein comprising human DNase1, human RNase1, and a mutant human IgG1 Fc, wherein the human DNase1 is operably linked via a linker (e.g., a gly-ser linker) to human RNase1, from N-terminus to C terminus, and wherein the human RNase1 is operably linked via a linker to the mutant human IgG1 Fc domain wherein the mutant human IgG1 has a mutant hinge region (e.g., a cysteine substitution, such as with serine, e.g., SCC), and one or more CH2 mutations to reduce Fc7 receptor binding (e.g., P238S, P331S or both P238S and P331S, numbering according to EU index). In one embodiment, the optimized binuclease fusion protein comprises human DNase1 operably linked via a peptide linker (e.g., a gly-ser linker) to human RNase1 (N-terminus-DNase1-linker-RNase1-C terminus) and the human RNase1 is operably linked via a peptide linker (e.g., a gly-ser linker) to a mutant human IgG1 Fc domain having a mutant hinge region, SCC hinge, and P238S and P331S mutations. In yet another embodiment, the Fc domain further includes a mutation at a site of N-linked glycosylation, such as a substitution at N297 (numbering by Kabat).

In some embodiments, the optimized binuclease fusion protein further includes a first linker domain, and the first nuclease domain is operably coupled to the second nuclease domain, via the first linker domain.

In some embodiments, the optimized binuclease fusion protein further includes a second linker domain, and the second nuclease domain is operably coupled to Fc domain, via the second linker domain.

In some embodiments, the RNase domain is a wild-type RNase, such as wild-type human RNase1. In other embodiments, the RNase domain is a mutant RNase, such as an aglycosylated, underglycosylated, or deglycosylated RNase 1, such as human RNase1 N34S/N76S/N88S (SEQ ID NO: 28). In some embodiments, the RNase containing optimized binuclease fusion protein degrades circulating RNA and RNA in immune complexes, or inhibits interferon-alpha production, or both. In yet other embodiments, the activity of the RNase is not less than about 10-fold less, such as 9-fold less, 8-fold less, 7-fold less, 6-fold less, 5-fold less, 4-fold less, 3-fold less, or 2-fold less than the activity of a control RNase molecule. In yet other embodiments, the activity of the RNase is about equal to the activity of a control RNase molecule.

In some embodiments, the DNase domain is wild type DNase, such as wild type, human DNase1. In other embodiments, the DNase domain is a mutant DNase domain, such as mutant, human DNase1 A114F (SEQ ID NO: 21) or an aglycosylated, underglycosylated, or deglycosylated human DNase, such as mutant, human DNase1 N18S/N106S/A114F (SEQ ID NO: 24). In some embodiments, the DNase containing optimized binuclease fusion protein degrades circulating DNA and DNA in immune complexes, or inhibits interferon-alpha production, or both. In yet other embodiments, the activity of the DNase is not less than about 10-fold less, such as 9-fold less, 8-fold less, 7-fold less, 6-fold less, 5-fold less, 4-fold less, 3-fold less, or 2-fold less than the activity of a control DNase molecule. In yet other embodiments, the activity of the DNase is about equal to the activity of a control DNase molecule.

In some embodiments, the optimized binuclease fusion protein has a gly-ser linker separating the first and second nuclease domains, and/or the second nuclease domain from the Fc domain.

In some embodiments, the optimized binuclease fusion protein has an increased serum half-life and/or activity relative to a molecule that does not contain the Fc domain.

In some aspects, the optimized binuclease fusion protein may include the mutant, human DNase1 A114F domain set forth in SEQ ID NO: 21. In another embodiment, the optimized binuclease fusion protein may include the mutant, human DNase1 N18S/N106S/A114F domain set forth in SEQ ID NO: 24. In some embodiments, the DNase domain is mutant human DNase1 E13R/N74K/A114F/T205K (SEQ ID NO: 25). In other embodiments, the DNase domain is mutant human DNase1 E13R/N74K/A114F/T205K/N18S/N106S (SEQ ID NO: 26).

In some embodiments, DNase1 and RNase1 domains are aglycosylated, underglycosylated, or deglycosylated. In some embodiments, the DNase domain is a mutant DNase domain, such as mutant, human DNase1 and an aglycosylated, underglycosylated, or deglycosylated DNase domain, such as an aglycosylated, underglycosylated, or deglycosylated human DNase1. In one embodiment, the human DNase1 includes an alteration (e.g., a substitution) at one or more sites of N-linked glycosylation, such as N18 and N106 and at least one additional mutation selected from A114, E13, N74, T205, and combinations thereof. In another embodiment, the human DNase1 includes an alteration (e.g., a substitution) at N18, N106, or both N18 and N106 and an additional alertation (e.g., a substitution) at A114, E13, N74, T205, and combinations thereof. In yet another embodiment, the human DNase1 includes an alteration at N18, N106, A114, E13, N74 and T205, such as a substitution, e.g., N18S/N106S/A114F/E13R/N74K/T205K (SEQ ID NO: 26). In another embodiment, the optimized binuclease fusion protein with altered glycosylation includes the human, wild-type RNase1 domain set forth in SEQ ID NO: 27 In another embodiment, the optimized binuclease fusion protein with altered glycosylation includes the human, mutant RNase1 N34S/N76S/N88S domain set forth in SEQ ID NO: 28.

In some aspects, the invention provides an optimized binuclease fusion protein comprising the polypeptides having an amino acid sequence set forth in SEQ ID NOs: 1-17. In other aspects, the optimized binuclease fusion protein have an amino acid sequence at least 90% identical or at least 95% identical to an amino acid sequence set forth in SEQ ID NOs: 1-17.

In some aspects, the optimized binuclease fusion protein comprises a polypeptide comprising a first nuclease domain, a second nuclease domain and an Fc domain, wherein the first nuclease domain is DNase1 and the second nuclease domain is RNase1, wherein the DNase1 is operably linked with or without a linker in tandem from N- to C-terminus to the RNase1, and the RNase1 is operably linked with or without a linker to the Fc region. In some aspects, the RNase 1 is operably linked to the N-terminus of the Fc domain without a linker. In some aspects, the RNase 1 is operably linked to the C-terminus of the Fc domain without a linker. In some aspects, the DNase1 is operably linked to the RNase1 via a linker. In some aspects, the polypeptide compriese an amino acid sequence set forth in SEQ ID NO: 1 or SEQ ID NO:2, or a tandem binuclease fusion protein comprising an amino acid sequence at least 90% identical to the amino acid sequence set forth in SEQ ID NO:1 or SEQ ID NO:2. In some aspects, the tamdem binuclease fusion protein is a homodimer comprising any of the foregoing polypeptides.

In some aspects, the optimized binuclease fusion protein is a heterodimer comprising a first nuclease domain, a second nuclease domain, a first Fc domain and a second Fc domain, wherein the first nuclease domain is DNase1 and the second nuclease domain is RNase1, wherein the DNase1 is operably linked with or without a linker to the N- or C-terminus of the first Fc domain, and the RNase1 is operably linked with or without a linker to the N- or C-terminus of the second Fc domain. In some aspects of the foregoing heterodimer, the DNase 1 is operably linked without a linker to the N-terminus of the first Fc domain and the RNase1 is operably linked without a linker to the N-terminus of the second Fc domain.

In some aspects, the DNase 1 is operably linked with a linker to the N-terminus of the first Fc domain and the RNase1 is operably linked with a linker to the N-terminus of the second Fc domain. In some aspects, the DNase 1 is operably linked with a linker to the N-terminus of the first Fc domain and the RNase1 is operably linked without a linker to the C-terminus of the second Fc domain. In some aspects, the DNase 1 is operably linked without a linker to the N-terminus of the first Fc domain and the RNase1 is operably linked without a linker to the C-terminus of the second Fc domain. In some aspects the DNase 1 is operably linked with a linker to the N-terminus of the first Fc domain and the RNase1 is operably linked with a linker to the C-terminus of the second Fc domain. In some aspects, the DNase 1 is operably linked with a linker to the C-terminus of the first Fc domain and the RNase1 is operably linked with a linker to the C-terminus of the second Fc domain. In some aspects, the DNase 1 is operably linked without a linker to the C-terminus of the first Fc domain and the RNase1 is operably linked without a linker to the C-terminus of the second Fc domain. In some aspects, the DNase 1 is operably linked with a linker to the C-terminus of the first Fc domain and the RNase1 is operably linked without a linker to the N-terminus of the second Fc domain. In some aspects, the DNase 1 is operably linked with a linker to the C-terminus of the first Fc domain and the RNase1 is operably linked with a linker to the N-terminus of the second Fc domain.

In some aspects, the optimized binuclease fusion protein is a heterodimer comprising a first and second polypeptide sequence selected from the group consisting of:
(i) a first polypeptide comprising an amino acid sequence set forth in SEQ ID NO: 3, or a polypeptide comprising an amino acid sequence at least 90% identical to the amino acid sequence set forth in SEQ ID NO:3; and a second polypeptide comprising an amino acid sequence set forth in SEQ ID NO: 4, or a polypeptide comprising an amino acid sequence at least 90% identical to the amino acid sequence set forth in SEQ ID NO: 4, or
(ii) a first polypeptide comprising an amino acid sequence set forth in SEQ ID NO: 7, or a polypeptide comprising an amino acid sequence at least 90% identical to the amino acid sequence set forth in SEQ ID NO:7; and a second polypeptide comprising an amino acid sequence set forth in SEQ ID NO: 8, or a polypeptide comprising an amino acid sequence at least 90% identical to the amino acid sequence set forth in SEQ ID NO:8, or
(iii) a first polypeptide comprising an amino acid sequence set forth in SEQ ID NO:9, or a polypeptide comprising an amino acid sequence at least 90% identical to the amino acid sequence set forth in SEQ ID NO:9; and a second polypeptide comprising an amino acid sequence set forth in SEQ ID NO: 10, or a polypeptide comprising an amino acid sequence at least 90% identical to the amino acid sequence set forth in SEQ ID NO:10, or
(iv) a first polypeptide comprising an amino acid sequence set forth in SEQ ID NO:11, or a polypeptide comprising an amino acid sequence at least 90% identical to the amino acid sequence set forth in SEQ ID NO:11; and a second polypeptide comprising an amino acid sequence set forth in SEQ ID NO: 12, or a polypeptide comprising an amino acid sequence at least 90% identical to the amino acid sequence set forth in SEQ ID NO:12, or
(v) a first polypeptide comprising an amino acid sequence set forth in SEQ ID NO:15, or a polypeptide comprising an amino acid sequence at least 90% identical to the amino acid sequence set forth in SEQ ID NO:15; and a second polypeptide comprising an amino acid sequence set forth in SEQ ID NO:16, or a polypeptide comprising an amino acid sequence at least 90% identical to the amino acid sequence set forth in SEQ ID NO:16.

Other aspects relate to a heterodimer comprising a first nuclease domain, a second nuclease domain and a first Fc domain and a second Fc domain, wherein the first nuclease domain is DNase1 and the second nuclease domain is RNase1, wherein
(i) the DNase1 is operably linked with or without a linker to the N-terminus of the first Fc domain, and the RNase1 is operably linked with or without a linker to the C-terminus of the first Fc domain, or
(ii) the RNase1 is operably linked with or without a linker to the N-terminus of the first Fc domain, and the DNase1 is operably linked with or without a linker to the C-terminus of the first Fc domain.

In some aspects of the foregoing heterodimer, the DNase 1 is operably linked without a linker to the N-terminus of the first Fc domain and the RNase1 is operably linked with a linker to the C-terminus of the first Fc domain. In some aspects, the DNase 1 is operably linked with a linker to the N-terminus of the first Fc domain and the RNase1 is operably linked with a without a linker to the C-terminus of the first Fc domain. In some aspects, the RNase 1 is operably linked without a linker to the N-terminus of the first Fc domain and the DNase 1 is operably linked with a linker to the C-terminus of the first Fc domain. In some aspects, the RNase 1 is operably linked with a linker to the N-terminus of the first Fc domain and the DNase 1 is operably linked with a linker to the C-terminus of the first Fc domain.

In some aspects, a heterodimer comprises a first and second polypeptide sequence selected from the group consisting of:
(i) a first polypeptide comprising an amino acid sequence set forth in SEQ ID NO:5, or a polypeptide comprising an amino acid sequence at least 90% identical to the amino acid sequence set forth in SEQ ID NO:5; and a second polypeptide comprising an amino acid sequence set forth in SEQ ID NO:6, or a polypeptide comprising an amino acid sequence at least 90% identical to the amino acid sequence set forth in SEQ ID NO:6, or
(ii) a first polypeptide comprising an amino acid sequence set forth in SEQ ID NO:13, or a polypeptide comprising an amino acid sequence at least 90% identical to the amino acid sequence set forth in SEQ ID NO:13; and a second polypeptide comprising an amino acid sequence set forth in SEQ ID NO:14, or a polypeptide comprising an amino acid sequence at least 90% identical to the amino acid sequence set forth in SEQ ID NO:14.

In some aspects, any of the foregoing heterodimers comprise one or more CH3 mutations in the Fc domains to preferentially form heterodimers. In some aspects, the heterodimer comprises a first Fc domain comprising CH3 mutations T350V, L351Y, F405A, and Y407V, and a second Fc domain comprising CH3 mutations T350V, T366L, K392L, T394W, numbering according to the EU index.

Other aspects of the disclosure relate to compositions comprising any of the foregoing heterodimer and a pharmaceutically acceptable carrier. Nucleic acid molecules encoding the foregoing heterodimers, recombinant expression vectors and host cell transformed with the recombinant expression vectors, as well as methods of making the foregoing heterodimers are also disclosed.

Also disclosed herein is a method of making a tandem optimized binuclease fusion protein disclosed herein involving providing a host cell comprising a nucleic acid sequence that encodes the optimized binuclease fusion protein; and maintaining the host cell under conditions in which the optimized binuclease fusion protein is expressed.

Also disclosed herein is a method for treating or preventing a condition associated with an abnormal immune response by administering to a patient in need thereof an effective amount of a optimized binuclease fusion protein disclosed herein. In some embodiments, the condition is an autoimmune disease. In some embodiments, the autoimmune disease is selected from the group consisting of insulin-dependent diabetes mellitus, multiple sclerosis, experimental autoimmune encephalomyelitis, rheumatoid arthritis, experimental autoimmune arthritis, myasthenia gravis, thyroiditis, an experimental form of uveoretinitis, Hashimoto's thyroiditis, primary myxoedema, thyrotoxicosis, pernicious anaemia, autoimmune atrophic gastritis, IgG4 related disease, Addison's disease, premature menopause, male infertility, juvenile diabetes, Goodpasture's syndrome, pemphigus vulgaris, pemphigoid, sympathetic ophthalmia, phacogenic uveitis, autoimmune haemolytic anaemia, idiopathic leucopenia, primary biliary cirrhosis, active chronic hepatitis Hbs-ve, cryptogenic cirrhosis, ulcerative colitis, Sjogren's syndrome, scleroderma, Wegener's granulomatosis, polymyositis, dermatomyositis, discoid LE, systemic lupus erythematosus (SLE), and connective tissue disease. In some embodiments, the autoimmune disease is SLE or Sjogren's syndrome.

Also disclosed herein is a method of treating SLE or Sjogren's syndrome comprising administering to a subject a optimized binuclease fusion protein containing composition in an amount effective to degrade immune complexes containing RNA, DNA or both RNA and DNA. In some aspects, the composition includes a pharmaceutically acceptable carrier and a optimized binuclease fusion protein as described herein. In other aspects, the composition includes a optimized binuclease fusion protein having an amino acid sequence set forth in SEQ ID NO: 1.

In another aspect, the invention relates to optimized binuclease fusion proteins for use in treating diseases characterized by defective clearance or processing of apoptotic cells and cell debris, such as SLE. In some embodiments, the optimized binuclease fusion protein comprises amino acid sequences set forth in SEQ ID NOs: 4 and 5.

In another aspect, the invention relates to the use of the optimized binuclease fusion proteins for manufacturing a medicament for treating diseases characterized by defective clearance or processing of apoptotic cells and cell debris, such as SLE. In some embodiments, the optimized binuclease fusion protein comprises amino acid sequences set forth in SEQ ID NOs: 5 and 6.

In another aspect, the invention relates to the use of the optimized binuclease fusion proteins for manufacturing a medicament for treating diseases characterized by defective clearance or processing of apoptotic cells and cell debris, such as SLE. In some embodiments, the optimized binuclease fusion protein comprises amino acid sequences set forth in SEQ ID NOs: 7 and 8.

In another aspect, the invention relates to the use of the optimized binuclease fusion proteins for manufacturing a medicament for treating diseases characterized by defective clearance or processing of apoptotic cells and cell debris, such as SLE. In some embodiments, the optimized binuclease fusion protein comprises amino acid sequences set forth in SEQ ID NOs: 13 and 14.

In another aspect, the invention relates to the use of the optimized binuclease fusion proteins for manufacturing a medicament for treating diseases characterized by defective clearance or processing of apoptotic cells and cell debris, such as SLE. In some embodiments, the optimized binuclease fusion protein comprises amino acid sequences set forth in SEQ ID NOs: 15 and 16.

### BRIEF DESCRIPTION OF THE DRAWINGS

These and other features, aspects, and advantages of the present invention will become better understood with regard to the following description, and accompanying drawing, where:

FIG. 1 is a depiction of exemplary optimized binuclease fusion proteins.

FIG. 2 is a graph showing RNase activity as measured by OD₂₆₀.

FIG. 3 shows DNase activity as measured by OD₆₂₀ (left) and IC₅₀ (right).

### DETAILED DESCRIPTION

Systemic lupus erythematosus (SLE) is a multisystem autoimmune disease characterized by the presence of high titer autoantibodies directed against self nucleoproteins. There is strong evidence that defective clearance or processing of dead and dying cells in SLE leads to disease, predominantly through accumulation of ribo- and deoxy-ribonucleoproteins (abbreviated nucleoproteins). The nucleoproteins cause damage through three mechanisms: i) activation of the innate immune system to produce inflammatory cytokines; ii) serve as antigens to generate circulating immune complexes; and iii) serve as antigens to generate in situ complex formation at local sites such as the kidney.

The present invention provides methods for treating diseases characterized by defective clearance or processing of apoptotic cells and cell debris, such as SLE and Sjogren's syndrome, by administering an effective amount of a long-acting nuclease activity to degrade extracellular RNA and DNA containing immune complexes. Such treatment can inhibit production of Type I interferons (IFNs) which are prominent cytokines in SLE and are strongly correlated with disease activity and nephritis.

The present invention relates, in part, to the provision of such long-acting nucleases. In particular, the invention relates to an optimized binuclease fusion protein, such as a tandem binuclease fusion protein comprising a first nuclease domain, a second nuclease domain and an Fc region, wherein the first nuclease domain is DNase1 and the second nuclease domain is RNase1, wherein the DNase1 is operably linked with or without a linker in tandem from N- to C-terminus to the RNase1, and the RNase1 is operably linked to the N- or C-terminus of an Fc region.

In other embodiments, the invention relates to an optimized binuclease fusion protein, such as a heterodimeric binuclease fusion protein comprising a first nuclease domain, a second nuclease domain and an Fc region, wherein the first nuclease domain is DNase1 and the second nuclease domain is RNase1, wherein the DNase1 is operably linked with or without a linker to the N- or C-terminus of an Fc region, and the RNase1 is operably linked with or without a linker to the N- or C-terminus of an Fc region, thereby forming a heterodimer.

In some aspects, the optimized binuclease fusion protein exhibits enhanced pharmacokinetic activity relative to the either first or second nuclease domain alone. Such optimized binuclease fusion proteins exhibit altered, e.g., improved, serum half-life relative to either the first or second nuclease domain alone.

In some aspects, the invention provides a optimized binuclease fusion protein comprising human DNase1, human RNase1, and a mutant human IgG1 Fc, wherein the human DNase1 is operably linked via a linker (e.g., a gly-ser linker) to human RNase1, from N-terminus to C terminus, and wherein the human RNase1 is operably linked via a linker to the mutant human IgG1 Fc domain wherein the mutant human IgG1 has a mutant hinge region (e.g., a cysteine substitution, such as with serine, e.g., SCC), and one or more CH2 mutations to reduce Fcγ receptor binding (e.g., P238S, P331S or both P238S and P331S, numbering according to EU index). In one embodiment, the optimized binuclease fusion protein comprises human DNase1 operably linked via a peptide linker (e.g., a gly-ser linker) to human RNase1 (N-terminus-DNase1-linker-RNase1-C terminus) and the human RNase1 is operably linked via a peptide linker (e.g., a gly-ser linker) to a mutant human IgG1 Fc domain having a mutant hinge region, SCC hinge, and P238S and P331S mutations.

Accordingly, in one embodiment, a subject with a disease characterized by defective clearance or processing of apoptotic cells and cell debris is treated by administering a optimized binuclease fusion protein, which includes both DNase1 and RNase1, such that the optimized binuclease fusion protein has increased bioavailability and/or serum half-life relative to the non-conjugated nuclease domains.

### Definitions

Terms used in the claims and specification are defined as set forth below unless otherwise specified.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, larger “peptide insertions” can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

“Polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res 1991; 19:5081; Ohtsuka et al., JBC 1985; 260:2605-8); Rossolini et al., Mol Cell Probes 1994; 8:91-8). For arginine and leucine, modifications at the second base can also be conservative. The term nucleic acid is used interchangeably with gene, cDNA, and mRNA encoded by a gene.

Polynucleotides of the present invention can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

As used herein, the term “operably linked” or “operably coupled” refers to a juxtaposition wherein the components described are in a relationship permitting them to function in their intended manner.

As used herein, the term “glycosylation” or “glycosylated” refers to a process or result of adding sugar moieties to a molecule (e.g., an optimized binuclease fusion protein).

As used herein, the term “altered glycosylation” refers to a molecule that is aglycosylated, deglycosylated, or underglycosylated.

As used herein, “glycosylation site(s)” refers to both sites that potentially could accept a carbohydrate moiety, as well as sites within the protein on which a carbohydrate moiety has actually been attached and includes any amino acid sequence that could act as an acceptor for an oligosaccharide and/or carbohydrate.

As used herein, the term “aglycosylation” or “aglycosylated” refers to the production of a molecule (e.g., an optimized binuclease fusion protein) in an unglycosylated form (e.g., by engineering an optimized binuclease fusion protein to lack amino acid residues that serve as acceptors of glycosylation). Alternatively, the optimized binuclease fusion protein can be expressed in, e.g., E. coli, to produce an aglycosylated optimized binuclease fusion protein.

As used herein, the term “deglycosylation” or “deglycosylated” refers to the process or result of enzymatic removal of sugar moieties on a molecule.

As used herein, the term “underglycosylation” or “underglycosylated” refers to a molecule in which one or more carbohydrate structures that would normally be present if produced in a mammalian cell has been omitted, removed, modified, or masked.

As used herein, the term “Fc region” and “Fc domain” is the portion of a native immunoglobulin formed by the respective Fc domains (or Fc moieties) of its two heavy chains without the variable regions which bind antigen. In some embodiments, an Fc domain begins in the hinge region just upstream of the papain cleavage site and ending at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In other embodiments, an Fc domain comprises a complete Fc domain (i.e., a hinge domain, a CH2 domain, and a CH3 domain). In one embodiment, an Fc domain comprises a hinge domain (or portion thereof) fused to a CH3 domain (or portion thereof). In another embodiment, an Fc domain comprises a CH2 domain (or portion thereof) fused to a CH3 domain (or portion thereof). In another embodiment, an Fc domain consists of a CH3 domain or portion thereof. In another embodiment, an Fc domain consists of a hinge domain (or portion thereof) and a CH3 domain (or portion thereof). In another embodiment, an Fc domain consists of a CH2 domain (or portion thereof) and a CH3 domain. In another embodiment, an Fc domain consists of a hinge domain (or portion thereof) and a CH2 domain (or portion thereof). In one embodiment, an Fc domain lacks at least a portion of a CH2 domain (e.g., all or part of a CH2 domain). In one embodiment, an Fc domain of the invention comprises at least the portion of an Fc molecule known in the art to be required for FcRn binding. In one embodiment, an Fc domain of the invention comprises at least the portion of an Fc molecule known in the art to be required for Protein A binding. In one embodiment, an Fc domain of the invention comprises at least the portion of an Fc molecule known in the art to be required for protein G binding. An Fc domain herein generally refers to a polypeptide comprising all or part of the Fc domain of an immunoglobulin heavy-chain. This includes, but is not limited to, polypeptides comprising the entire CHI, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As with Fc variants and native Fc's, the term Fc domain includes molecules in monomeric or multimeric form, whether digested from whole antibody or produced by other means.

As set forth herein, it will be understood by one of ordinary skill in the art that any Fc domain may be modified such that it varies in amino acid sequence from the native Fc domain of a naturally occurring immunoglobulin molecule.

The Fe domains of an optimized binuclease fusion protein of the disclosure may be derived from different immunoglobulin molecules. For example, an Fc domain of an optimized binuclease fusion protein may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule. The wild type human IgG1 Fc domain has the amino acid sequence set forth in SEQ ID NO: 45.

As used herein, the term “serum half-life” refers to the time required for the in vivo serum optimized binuclease fusion protein concentration to decline by 50%. The shorter the serum half-life of the optimized binuclease fusion protein, the shorter time it will have to exert a therapeutic effect.

As used herein, the term “optimized binuclease fusion protein” refers to polypeptides that comprise at least two nuclease domains operably linked, with or without a linker, to an Fc domain, or a variant or fragment thereof, and nucleic acids encoding such polypeptides. In some embodiments, an optimized binuclease fusion protein is a tandem binuclease fusion protein, e.g., a one or more DNase 1 domains and one or more RNase 1 domains linked in tandem to either the N- or C-terminus of one or more Fc domain. In some embodiments, an optimized binuclease fusion protein is a heterodimeric binuclease fusion protein.

As used herein, the term “tandem binuclease fusion protein” refers to a polypeptide that comprises at least two nuclease domains linked in tandem (from N- to C-terminus) and an Fc domain, or a variant or fragment thereof, and nucleic acids encoding such polypeptides. For example, in one embodiment, a tandem binuclease fusion protein is a polypeptide comprising at least one DNase1 domain and at least one RNase1 domain operably linked in tandem to at least one Fc domain. As another example, a tandem binuclease fusion protein includes from N- to C-terminus a DNase1 domain, a first linker, an RNase1 domain, a second linker, and an Fc domain, or a variant or fragment thereof.

As used herein, the term “heterodimeric binuclease fusion protein” refers to a heterodimer comprising a first and a second polypeptide, which together comprise at least two nuclease domains and two Fc domains, variants or fragment thereof, and nucleic acids encoding such polypeptides. In some embodiments, a heterodimeric binuclease fusion protein is a heterodimer comprising at least one DNase1 domain and at least one RNase1 domain operably linked to at least one Fc domain, wherein the DNase 1 domain is operably linked with or without a linker to the N- or C-terminus of a first Fc domain and an RNase1 domain is operably linked with or without a linker the N- or C-terminus of a same (first Fc domain) or a different Fc domain (second Fc domain), such that the DNase 1 domain and the RNase 1 domain are located on opposite ends (N- or C-terminus) of either the same (first Fc domain) or different Fc domain (second Fc domain). In some embodiments, the heterodimer comprises a DNase 1 domain operably linked with or without a linker to the N- or C-terminus of a first Fc domain, and a RNase 1 operably linked with or without a linker to the N- or C-terminus of the second Fc domain, such that the DNase 1 and RNase 1 domains are located at the same end (N- or C-terminus) of the heterodimer in tandem. In some embodiments, the heterodimer comprises a DNase 1 domain operably linked with or without a linker to the N-terminus of a first Fc domain, and a RNase 1 operably linked with or without a linker to the C-terminus of the first Fc domain. In some embodiments, the RNase1 is operably linked with or without a linker to the N-terminus of the first Fc domain, and the DNase1 is operably linked with or without a linker to the C-terminus of the first Fc domain.

As used herein, the term “variant” refers to a polypeptide derived from a wild-type nuclease or Fc domain and differs from the wild-type by one or more alteration(s), i.e., a substitution, insertion, and/or deletion, at one or more positions. A substitution means a replacement of an amino acid occupying a position with a different amino acid. A deletion means removal of an amino acid occupying a position. An insertion means adding 1 or more, such as 1-3 amino acids, immediately adjacent to an amino acid occupying a position. Variant polypeptides necessarily have less than 100% sequence identity or similarity with the wild-type polypeptide. In some embodiments, the variant polypeptide will have an amino acid sequence from about 75% to less than 100% amino acid sequence identity or similarity with the amino acid sequence of wild-type polypeptide, or from about 80% to less than 100%, or from about 85% to less than 100%, or from about 90% to less than 100% (e.g., 91%, 92%, 93%, 94%, 95%, 96%, 97%, 98% o, 99%) or from about 95% to less than 100%, e.g., over the length of the variant polypeptide.

In certain aspects, the optimized binuclease fusion proteins employ one or more “linker domains,” such as polypeptide linkers. As used herein, the term “linker domain” refers to one or more amino acids which connect two or more peptide domains in a linear polypeptide sequence. As used herein, the term “polypeptide linker” refers to a peptide or polypeptide sequence (e.g., a synthetic peptide or polypeptide sequence) which connects two or more polypeptide domains in a linear amino acid sequence of a protein. For example, polypeptide linkers may be used to operably link a first and second nuclease domain to each other, or a first or second nuclease domain to an Fc domain. Such polypeptide linkers in some embodiments provide flexibility to the polypeptide molecule. In some embodiments the polypeptide linker is used to connect (e.g., genetically fuse) a DNase1 to an RNase1 and/or RNase1 to an Fc domain. An optimized binuclease fusion protein may include more than one linker domain or peptide linker. Various peptide linkers are known in the art.

As used herein, the term “gly-ser polypeptide linker” refers to a peptide that consists of glycine and serine residues. An exemplary gly/ser polypeptide linker comprises the amino acid sequence (Gly₄Ser)n. In some embodiments, n is 1 or more, such as 2 or more, 3 or more, 4 or more, 5 or more, 6 or more, 7 or more, 8 or more, 9 or more, or 10 or more (e.g., (Gly₄Ser)10). Another exemplary gly/ser polypeptide linker comprises the amino acid sequence Ser(Gly₄Ser)n. In some embodiments, n is 1 or more, such as 2 or more, 3 or more, 4 or more, 5 or more, 6 or more, 7 or more, 8 or more, 9 or more, or 10 or more (e.g., Ser(Gly₄Ser)10).

As used herein, the terms “coupled,” “linked,” “fused,” or “fusion,” are used interchangeably. These terms refer to the joining together of two more elements or components or domains, by whatever means including chemical conjugation or recombinant means. Methods of chemical conjugation (e.g., using heterobifunctional crosslinking agents) are known in the art.

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. Polypeptides derived from another peptide may have one or more mutations relative to the starting polypeptide, e.g., one or more amino acid residues which have been substituted with another amino acid residue or which has one or more amino acid residue insertions or deletions.

In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity.

In one embodiment, a polypeptide of the disclosure consists of, consists essentially of, or comprises an amino acid sequence as set forth in the Sequence Listing or Sequence Table disclosed herein and functionally active variants thereof. In an embodiment, a polypeptide includes an amino acid sequence at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to an amino acid sequence set forth in the Sequence Listing or Sequence Table disclosed herein. In some embodiments, a polypeptide includes a contiguous amino acid sequence at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to a contiguous amino acid sequence set forth in the Sequence Listing or Sequence Table disclosed herein. In some embodiments, a polypeptide includes an amino acid sequence having at least 10, such as at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 200, at least 300, at least 400, or at least 500 (or any integer within these numbers) contiguous amino acids of an amino acid sequence set forth in Sequence Listing or Sequence Table disclosed herein.

In some embodiments, the optimized binuclease fusion proteins of the disclosure are encoded by a nucleotide sequence. Nucleotide sequences of the disclosure can be useful for a number of applications, including: cloning, gene therapy, protein expression and purification, mutation introduction, DNA vaccination of a host in need thereof, antibody generation for, e.g., passive immunization, PCR, primer and probe generation, siRNA design and generation (see, e.g., the Dharmacon siDesign website), and the like. In some embodiments, the nucleotide sequence of the disclosure comprises, consists of, or consists essentially of, a nucleotide sequence that encodes the amino acid sequence of the optimized binuclease fusion proteins selected from the Sequence Table or Sequence Listing. In some embodiments, a nucleotide sequence includes a nucleotide sequence at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to a nucleotide sequence encoding an amino acid sequence of the Sequence Listing or Sequence Table disclosed herein. In some embodiments, a nucleotide sequence includes a contiguous nucleotide sequence at least 80%, such as at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to a contiguous nucleotide sequence encoding an amino acid sequence set forth in the Sequence Listing or Sequence Table disclosed herein. In some embodiments, a nucleotide sequence includes a nucleotide sequence having at least 10, such as at least 15, such as at least 20, at least 25, at least 30, at least 35, at least 40, at least 45, at least 50, at least 55, at least 60, at least 65, at least 70, at least 75, at least 80, at least 85, at least 90, at least 95, at least 100, at least 200, at least 300, at least 400, or at least 500 (or any integer within these numbers) contiguous nucleotides of a nucleotide sequence encoding an amino acid sequence set forth in the Sequence Listing or Sequence Table disclosed herein.

It will also be understood by one of ordinary skill in the art that the optimized binuclease fusion proteins may be altered such that they vary in sequence from the naturally occurring or native sequences from which their components (e.g., nuclease domains, linker domains, and Fc domains) are derived, while retaining the desirable activity of the native sequences. For example, nucleotide or amino acid substitutions leading to conservative substitutions or changes at “non-essential” amino acid residues may be made. An isolated nucleic acid molecule encoding a non-natural variant can be created by introducing one or more nucleotide substitutions, additions or deletions into the nucleotide sequence of the optimized binuclease fusion protein such that one or more amino acid substitutions, additions or deletions are introduced into the encoded protein. Mutations may be introduced by standard techniques, such as site-directed mutagenesis and PCR-mediated mutagenesis.

The optimized binuclease fusion proteins may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Thus, a nonessential amino acid residue in an optimized binuclease fusion protein is preferably replaced with another amino acid residue from the same side chain family. In another embodiment, a string of amino acids can be replaced with a structurally similar string that differs in order and/or composition of side chain family members. Alternatively, in another embodiment, mutations may be introduced randomly along all or part of a coding sequence, such as by saturation mutagenesis, and the resultant mutants can be incorporated into the optimized binuclease fusion proteins and screened for their ability to bind to the desired target.

The term “ameliorating” refers to any therapeutically beneficial result in the treatment of a disease state, e.g., an autoimmune disease state (e.g., SLE, Sjogren's syndrome), including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” or “subject” or “patient” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv Appl Math 1981; 2:482, by the homology alignment algorithm of Needleman & Wunsch, J Mol Biol 1970; 48:443, by the search for similarity method of Pearson & Lipman, PNAS 1988; 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al, infra).

One example of an algorithm that is suitable for determining percent sequence identity and sequence similarity is the BLAST algorithm, which is described in Altschul et al., J Mol Biol 1990; 215:403-10. Software for performing BLAST analyses is publicly available through the National Center for Biotechnology Information website.

The term “sufficient amount” means an amount sufficient to produce a desired effect.

The term “therapeutically effective amount” is an amount that is effective to ameliorate a symptom of a disease. A therapeutically effective amount can be a “prophylactically effective amount” as prophylaxis can be considered therapy.

The term “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

It must be noted that, as used in the specification and the appended claims, the singular forms “a,” “an” and “the” include plural referents unless the context clearly dictates otherwise.

### Optimized Binuclease Fusion Proteins

The optimized binuclease fusion proteins of the disclosure include an Fc domain, or a variant or fragment thereof, that alters the serum half-life of the nuclease molecules to which it is fused compared to nuclease molecules that are not fused to the Fc domain, or a variant or fragment thereof.

In some embodiments, a composition of the disclosure includes an optimized binuclease fusion protein. In some embodiments, an optimized binuclease fusion protein includes a nuclease domain operably coupled to an Fc domain, or a variant or fragment thereof.

In some embodiments, the nuclease domain is operably coupled to the Fc domain, or a variant or fragment thereof, via a linker domain. In some embodiments, the linker domain is a linker peptide. In some embodiments, the linker domain is a linker nucleotide.

In some embodiments, the optimized binuclease fusion protein includes a leader molecule, e.g., a leader peptide. In some embodiments, the leader molecule is a leader peptide positioned at the N-terminus of the nuclease domain. In some embodiments, an optimized binuclease fusion protein of the invention comprises a leader peptide at the N-terminus of the molecule, wherein the leader peptide is later cleaved from the optimized binuclease fusion protein. Methods for generating nucleic acid sequences encoding a leader peptide fused to a recombinant protein are well known in the art. In some embodiments, any of the optimized binuclease fusion proteins of the present invention can be expressed either with or without a leader fused to their N-terminus. The protein sequence of an optimized binuclease fusion protein of the present disclosure following cleavage of a fused leader peptide can be predicted and/or deduced by one of skill in the art.

In some embodiments the leader is a VK3 leader peptide (VK3LP), wherein the leader peptide is fused to the N-terminus of the optimized binuclease fusion protein. Such leader sequences can improve the level of synthesis and secretion of the optimized binuclease fusion protein in mammalian cells. In some embodiments, the leader is cleaved, yielding optimized binuclease fusion proteins. In some embodiments, an optimized binuclease fusion protein of the present invention is expressed without a leader peptide fused to its N-terminus, and the resulting optimized binuclease fusion protein has an N-terminal methionine.

In some embodiments, the optimized binuclease fusion protein includes two nuclease domains operably coupled to each other in tandem and further operably coupled to the N- or C-terminus of the same or different Fc domains, or a variant or fragment thereof.

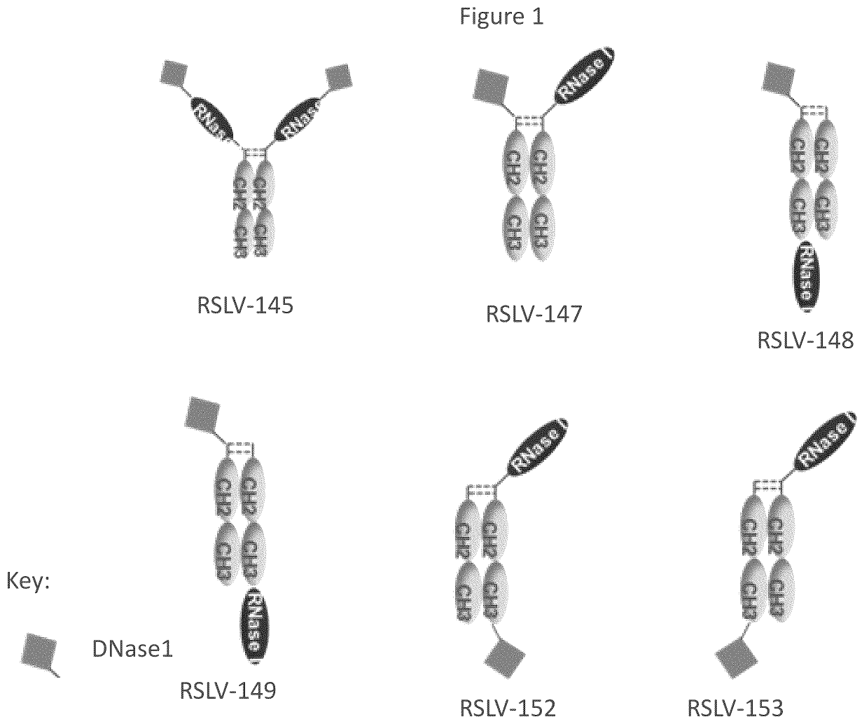

FIG. 1 displays exemplary configurations of the optimized binuclease fusion proteins, and the Sequence Table provides the sequences of exemplary optimized binuclease fusion proteins of various configurations.

In some embodiments, an optimized binuclease fusion protein is a multi-nuclease protein (e.g., both RNase and DNase or two RNA or DNA nucleases with different specificity for substrate) fused to the same or different Fc domains, or a variant or fragment thereof, that specifically binds to extracellular immune complexes.

In one embodiment, the nuclease domain is operably coupled (e.g., chemically conjugated or genetically fused (e.g., either directly or via a polypeptide linker)) to the N-terminus of a Fc domain, or a variant or fragment thereof. In another embodiment, the nuclease domain is operably coupled (e.g., chemically conjugated or genetically fused (e.g., either directly or via a polypeptide linker)) to the C-terminus of a Fc domain, or a variant or fragment thereof. In other embodiments, a nuclease domain is operably coupled (e.g., chemically conjugated or genetically fused (e.g., either directly or via a polypeptide linker)) via an amino acid side chain of a Fc domain, or a variant or fragment thereof.

In certain embodiments, the optimized binuclease fusion proteins of the disclosure comprise two or more nuclease domains and at least one Fc domain, or a variant or fragment thereof. For example, nuclease domains may be operably coupled to both the N-terminus and C-terminus of the same or different Fc domains, or variants or fragments thereof, with optional linkers between the nuclease domains and the Fc domain(s), variant(s) or fragment(s) thereof. In some embodiments, the nuclease domains are identical, e.g., RNase and RNase, or DNase1 and DNase1. In other embodiments, the nuclease domains are different, e.g., DNase and RNase.

In some embodiments, two or more nuclease domains are operably coupled to each other (e.g., via a polypeptide linker) in series, and the tandem array of nuclease domains is operably coupled (e.g., chemically conjugated or genetically fused (e.g., either directly or via a polypeptide linker)) to either the C-terminus or the N-terminus of the same or different Fc domains, or variants or fragments thereof. In other embodiments, the tandem array of nuclease domains is operably coupled to both the N-terminus and the C-terminus of the same Fc domain, or a variant or fragment thereof. In some embodiments, the nuclease domains are operably linked in tandem (e.g., N-DNase-RNase-C or N-RNase-DNase-C) with or without a linker to the N- or C-terminus of the same or different Fe domains. In some embodiments, the tandem binuclease fusion proteins form a homodimer or a heterodimer.

In other embodiments, one or more nuclease domains may be inserted between two Fe domains, or variants or fragments thereof. For example, one or more nuclease domains may form all or part of a polypeptide linker of a optimized binuclease fusion protein of the disclosure.

In some embodiments, the optimized binuclease fusion proteins comprise at least two nuclease domains (e.g., RNase and DNase), at least one linker domain, and at least one Fc domain, or a variant or fragment thereof.

In some embodiments, the optimized binuclease fusion proteins of the disclosure comprise a Fc domain, or a variant or fragment thereof, as described supra, thereby increasing serum half-life and bioavailability of the optimized binuclease fusion proteins.

In some embodiments, an optimized binuclease fusion protein comprises one or more polypeptides such as a polypeptide comprising an amino acid sequence as shown in any of SEQ ID NOs: 1-17.

It will be understood by the skilled artisan that other configurations of the nuclease domains and Fc domains are possible, with the inclusion of optional linkers between the nuclease domains and/or between the nuclease domains and Fc domain. It will also be understood that domain orientation can be altered, so long as the nuclease domains are active in the particular configuration tested.

In certain embodiments, the optimized binuclease fusion proteins of the disclosure have at least one nuclease domain specific for a target molecule which mediates a biological effect. In another embodiment, binding of the optimized binuclease fusion proteins of the disclosure to a target molecule (e.g. DNA or RNA) results in the reduction or elimination of the target molecule, e.g., from a cell, a tissue, or from circulation.

In other embodiments, the optimized binuclease fusion proteins of the disclosure may be assembled together or with other polypeptides to form binding proteins having two or more polypeptides (“multimers”), wherein at least one polypeptide of the multimer is an optimized binuclease fusion protein of the invention. Exemplary multimeric forms include dimeric, trimeric, tetrameric, and hexameric altered binding proteins and the like. In one embodiment, the polypeptides of the multimer are the same (i.e., homomeric altered binding proteins, e.g., homodimers, homotetramers). In another embodiment, the polypeptides of the multimer are different (e.g., heteromeric).

In some embodiments, an optimized binuclease fusion protein has a serum half-life that is increased at least about 1.5-fold, such as at least 3-fold, at least 5-fold, at least 10-fold, at least about 20-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1000-fold, or 1000-fold or greater relative to the corresponding nuclease molecules not fused to the Fc domain, or a variant or fragment thereof. In other embodiments, an optimized binuclease fusion protein has a serum half-life that is decreased at least about 1.5-fold, such as at least 3-fold, at least 5-fold, at least 10-fold, at least about 20-fold, at least about 50-fold, at least about 100-fold, at least about 200-fold, at least about 300-fold, at least about 400-fold, at least about 500-fold, or 500-fold or lower relative to the corresponding nuclease molecules not fused to the Fc domain, or a variant or fragment thereof. Routine art-recognized methods can be used to determine the serum half-life of optimized binuclease fusion proteins of the disclosure.

In some embodiments, the activity of the RNase in the optimized binuclease fusion protein is not less than about 10-fold less, such as 9-fold less, 8-fold less, 7-fold less, 6-fold less, 5-fold less, 4-fold less, 3-fold less, or 2-fold less than the activity of a control RNase molecule. In some embodiments, the activity of the RNase in the optimized binuclease fusion protein is about equal to the activity of a control RNase molecule.

In some embodiments, the activity of the DNase in the optimized binuclease fusion protein is not less than about 10-fold less, such as 9-fold less, 8-fold less, 7-fold less, 6-fold less, 5-fold less, 4-fold less, 3-fold less, or 2-fold less than the activity of a control DNase molecule. In some embodiments, the activity of the DNase in the optimized binuclease fusion protein is about equal to the activity of a control DNase molecule.

In some embodiments, the optimized binuclease fusion proteins can be active towards extracellular immune complexes containing DNA and/or RNA, e.g., either in soluble form or deposited as insoluble complexes.

In some embodiments, the activity of the optimized binuclease fusion protein is detectable in vitro and/or in vivo. In some embodiments, the optimized binuclease fusion protein binds to a cell, a malignant cell, or a cancer cell and interferes with its biologic activity.

In another aspect, a multifunctional RNase or DNase molecule is provided that is attached to another enzyme or antibody having binding specificity, such as an scFv targeted to RNA or DNA or a second nuclease domain with the same or different specificities as the first domain.

In some embodiments, linker domains include (gly4ser) 3, 4 or 5 variants that alter the length of the linker by 5 amino acid progressions. In another embodiment, a linker domain is approximately 18 amino acids in length and includes an N-linked glycosylation site, which can be sensitive to protease cleavage in vivo. In some embodiments, an N-linked glycosylation site can protect the optimized binuclease fusion proteins from cleavage in the linker domain. In some embodiments, an N-linked glycosylation site can assist in separating the folding of independent functional domains separated by the linker domain.

In some embodiments, the linker domain is an NLG linker (VDGASSPVNVSSPSVQDI) (SEQ ID NO: 41).

In some embodiments, the optimized binuclease fusion protein includes substantially all or at least an enzymatically active fragment of a DNase. In some embodiments, the DNase is a Type I secreted DNase, preferably a human DNase such as mature human pancreatic DNase 1 (UniProtKB entry P24855, SEQ ID NO: 20). In some embodiments, a naturally occurring variant allele, A114F (SEQ ID NO: 21), which shows reduced sensitivity to actin is included in a DNase1 optimized binuclease fusion protein (see Pan et al., JBC 1998; 273:18374-81; Zhen et al., BBRC 1997; 231:499-504; Rodriguez et al., Genomics 1997; 42:507-13). In other embodiments, a naturally occurring variant allele, G105R (SEQ ID NO: 22), which exhibits high DNase activity relative to wild type DNase1, is included in a DNase1 optimized binuclease fusion protein (see Yasuda et al., Int J Biochem Cell Biol 2010; 42:1216-25). In some embodiments, this mutation is introduced into an optimized binuclease fusion protein to generate a more stable derivative of human DNase1. In some embodiments, the DNase is human, wild type DNase1 or human, DNase1 A114F mutated to remove all potential N-linked glycosylation sites, i.e., asparagine residues at positions 18 and 106 of the DNase1 domain set forth in SEQ ID NO: 20 (i.e., human DNase1 N18S/N106S/A114F, SEQ ID NO: 24), which correspond to asparagine residues at positions 40 and 128, respectively, of full length pancreatic DNase1 with the native leader (SEQ ID NO: 23). In some embodiments, the DNase is a human DNase1 comprising one or more basic (i.e., positively charged) amino acid substitutions to increase DNase functionality and chromatin cleavage. In some embodiments, basic amino acids are introduced into human DNase1 at the DNA binding interface to enhance binding with negatively charged phosphates on DNA substrates (see U.S. Pat. Nos. 7,407,785; 6,391,607). This hyperactive DNase1 may be referred to as “chromatin cutter.”

In some embodiments, 1, 2, 3, 4, 5 or 6 basic amino acid substitutions are introduced into DNase1. For example, one or more of the following residues is mutated to enhance DNA binding: Gln9, Glu13, Thr14, His44, Asn74, Asn110, Thr205. In some embodiments one or more of the foregoing amino acids are substituted with basic amino acids such as, arginine, lysine and/or histidine. For example, a mutant human DNase can include one or more of the following substitutions: Q9R, E13R, T14K, H44K, N74K, N110R, T205K. In some embodiments, the mutant human DNase1 also includes an A114F substitution, which reduces sensitivity to actin (see U.S. Pat. No. 6,348,343). In one embodiment, the mutant human DNase1 includes the following substitutions: E13R, N74K, A114F and T205K.

In some embodiments, the mutant human DNase1 further includes mutations to remove potential glycosylation sites, e.g., asparagine residues at positions 18 and 106 of the DNase1 domain set forth in SEQ ID NO: 20, which correspond to asparagines residues at positions 40 and 128, respectively of full length pancreatic DNase1 with the native leader. In one embodiment, the mutant human DNase1 includes the following substitutions: E13R/N74K/A114F/T205K/N18S/N106S.

In some embodiments, the DNase is DNase 1-like (DNaseL) enzyme, 1-3 (UniProtKB entry Q13609; SEQ ID NO: 46). In some embodiments, the DNase is three prime repair exonuclease 1 (TREX1; UniProtKB entry Q9NSU2; SEQ ID NO: 47). In some embodiments, the DNase is DNase2. In some embodiments, the DNase2 is DNAse2 alpha (i.e., DNase2; UnitProtKB entry 000115SEQ ID NO: 48) or DNase2 beta (i.e., DNase2-like acid DNase; UnitProtKB entry Q8WZ79; SEQ ID NO: 49). In some embodiments, the N-linked glycosylation sites of DNase 1L3, TREX1, DNase2 alpha, or DNase2 beta are mutated such as to remove potential N-linked glycosylation sites. In some embodiments, a DNase-linker-Fc domain containing a 20 or 25 aa linker domain is made.

In some embodiments, the optimized binuclease fusion protein includes a RNase1, preferably human pancreatic RNase1 (UniProtKB entry P07998; SEQ ID NO: 27) of the RNase A family. In some embodiments, the human RNase1 is mutated to remove all potential N-linked glycosylation sites, i.e., asparagine residues at positions 34, 76, and 88 of the RNase1 domain set forth in SEQ ID NO: 27 (human RNase1 N34S/N76S/N88S, SEQ ID NO: 28), which correspond to asparagine residues at positions 62, 104, and 116, respectively, of full length pancreatic RNase1 with the native leader (SEQ ID NO: 29). In some embodiments, a RNase1-linker-Fc containing a 20 or 25 aa linker domain is made.

In some embodiments, optimized binuclease fusion proteins include DNase-linker-RNase-Fc, wherein the RNase1 domain is located at the COOH side of the Fc. In other embodiments, optimized binuclease fusion proteins include DNase-linker-RNase-Fc, wherein the RNase1 domain is located at the NH2 side of the Fc. In some embodiments, optimized binuclease fusion proteins include: DNase-Fc and RNase-Fc; DNase1-Fc-linker-RNase and Fc domain; DNase1-Fc and Fc-linker-RNase; Fc-linker-DNase1 and Fc-linker-RNase; RNase-Fc-linker-DNase and Fc domain; Fc-linker-DNase and Rnase-Fc; and RNase-Fc-linker-DNase.

In some embodiments, fusion junctions between enzyme domains and the other domains of the optimized binuclease fusion protein is optimized.

In some embodiments, the targets of the RNase enzyme activity of optimized binuclease fusion proteins are primarily extracellular, consisting of, e.g., RNA contained in immune complexes with anti-RNP autoantibody and RNA expressed on the surface of cells undergoing apoptosis. In some embodiments, the optimized binuclease fusion protein is active in the acidic environment of the endocytic vesicles. In some embodiments, an optimized binuclease fusion protein including a Fc domain, or a variant or fragment thereof, is adapted to be active both extracellularly and in the endocytic environment. In some aspects, this allows an optimized binuclease fusion protein including a wild-type Fc domain, or a variant or fragment thereof, to stop TLR7 signaling through previously engulfed immune complexes or by RNAs that activate TLR7 after viral infection. In some embodiments, the wild type RNase of an optimized binuclease fusion protein is not resistant to inhibition by an RNase cytoplasmic inhibitor. In some embodiments, the wild type RNase of an optimized binuclease fusion protein is not active in the cytoplasm of a cell.

In some embodiments, optimized binuclease fusion proteins include both DNase and RNase. In some embodiments, these optimized binuclease fusion proteins improve therapy of SLE because they digest or degrade immune complexes containing RNA, DNA, or a combination of both RNA and DNA, and are active extracellularly.

### Fe Domain

In some embodiments, the polypeptide comprising one or more nuclease domains is operably coupled to a Fc domain, which serves as a scaffold as well as a means to increase the serum half-life of the polypeptide. In some embodiments, the one or more nuclease domains and/or the Fc domain is aglycosylated, deglycosylated, or underglycosylated.

Suitable Fc domains are well-known in the art and include, but are not limited to, Fc and Fc variants, such as those disclosed in WO2011/053982, WO 02/060955, WO 02/096948, WO05/047327, WO05/018572, and US 2007/0111281 (the contents of the foregoing are incorporated herein by reference). It is within the abilities of the skilled artisan to use routine methods to introduce Fc domains (e.g., cloning, conjugation) into the optimized binuclease fusion proteins disclosed herein (with or without altered glycosylation).

In some embodiments, the Fc domain is a wild type human IgG1 Fc, such as is shown in SEQ ID NO: 45.

In some embodiments, an Fc domain is altered or modified, e.g., by mutation which results in an amino acid addition, deletion, or substitution. As used herein, the term “Fc domain variant” refers to an Fc domain having at least one amino acid modification, such as an amino acid substitution, as compared to the wild-type Fc from which the Fc domain is derived. For example, wherein the Fc domain is derived from a human IgG1 antibody, a variant comprises at least one amino acid mutation (e.g., substitution) as compared to a wild type amino acid at the corresponding position of the human IgG1 Fc region. The amino acid substitution(s) of an Fc variant may be located at a position within the Fc domain referred to as corresponding to the position number that that residue would be given in an Fc region in an antibody (numbering according to EU index).

In one embodiment, the Fc variant comprises one or more amino acid substitutions at an amino acid position(s) located in a hinge region or portion thereof. In another embodiment, the Fc variant comprises one or more amino acid substitutions at an amino acid position(s) located in a CH2 domain or portion thereof. In another embodiment, the Fc variant comprises one or more amino acid substitutions at an amino acid position(s) located in a CH3 domain or portion thereof. In another embodiment, the Fc variant comprises one or more amino acid substitutions at an amino acid position(s) located in a CH4 domain or portion thereof.

In some embodiments, the Fc region has a mutation at N83 (i.e., N297 by Kabat numbering), yielding an aglycosylated Fc region (e.g., Fc N83S; SEQ ID NO: 50). In some embodiments, the Fc domain includes mutations in one or more of the three hinge region cysteines (residues 220, 226, and 229, numbering according to the EU index). In some embodiments, one or more of the three hinge cysteines in the Fc domain can be mutated to SCC (SEQ ID NO: 51) or SSS (SEQ ID NO: 52), where in “S” represents an amino acid substitution of cysteine with serine. Accordingly “SCC” indicates an amino acid substitution to serine of only the first cysteine of the three hinge region cysteines (residues 220, 226, and 229, numbering according to the EU index), whereas “SSS” indicates that all three cysteines in the hinge region are substituted with serine (residues 220, 226, and 229, numbering according to the EU index).

In some aspects, the Fc domain is a mutant human IgG1 Fc domain. In some aspects, a mutant Fc domain comprises one or more mutations in the hinge, CH2, and/or CH3 domains.

### CH2 Substitutions

In some aspects, a mutant Fc domain includes a P238S mutation. In some aspects, a mutant Fc domain includes a P331S mutation. In some aspects, a mutant Fc domain includes a P238S mutation and a P331S mutation. In some aspects, a mutant Fc domain comprises P238S and/or P331S, and may include mutations in one or more of the three hinge cysteines (residues 220, 226, and 229), numbering according to the EU index. In some aspects, a mutant Fc domain comprises P238S and/or P331S, and/or one or more mutations in the three hinge cysteines (residues 220, 226, and 229), numbering according to the EU index. In some aspects, a mutant Fc domain comprises P238S and/or P331S, and/or mutations in a hinge cysteine to SCC or in the three hinge cysteines to SSS. In some aspects, a mutant Fc domain comprises P238S and P331S and mutations in at least one of the three hinge cysteines. In some aspects, a mutant Fc domain comprises P238S and P331S and SCC. In some aspects, a mutant Fc domain comprises P238S and P331S and SSS. In some aspects, a mutant Fc domain includes P238S and SCC or SSS. In some aspects, a mutant Fc domain includes P331S and SCC or SSS. (All numbering according to the EU index).

In some aspects, a mutant Fc domain includes a mutation at a site of N-linked glycosylation, such as N297, e.g., a substitution of asparagine for another amino acid such as serine, e.g., N297S. In some aspects, a mutant Fc domain includes a mutation at a site of N-linked glycosylation, such as N297, e.g., a substitution of asparagine for another amino acid such as serine, e.g., N297S and a mutation in one or more of the three hinge cysteines. In some aspects, a mutant Fc domain includes a mutation at a site of N-linked glycosylation, such as N297, e.g., a substitution of asparagine for another amino acid such as serine, e.g., N297S and mutations in one of the three hinge cysteines to SCC or all three cysteines to SSS. In some aspects, a mutant Fc domain includes a mutation at a site of N-linked glycosylation, such as N297, e.g., a substitution of asparagine for another amino acid such as serine, e.g., N297 and one or more mutations in the CH2 domain which decrease FcγR binding and/or complement activation, such as mutations at P238 or P331 or both, e.g., P238S or P331S or both P238S and P331S. In some aspects, such mutant Fc domains can further include a mutation in the hinge region, e.g., SCC or SSS. (All numbering according to the EU index.) In some aspects, the mutant Fc domain is as shown in the Sequence Table or Sequence Listing herein.

### CH3 Substitutions

Heterodimers can be preferentially formed by mutations in the CH3 domain of the Fc domain on the heterodimeric binuclease fusion proteins disclosed herein. Heavy chains were first engineered for heterodimerization using a “knobs-into-holes” strategy (Rigway B, et al., Protein Eng., 9 (1996) pp. 617-621). The term “knob-into-hole” refers to the technology directing the pairing of two polypeptides together in vitro or in vivo by introducing a pertuberance (knob) into one polypeptide and a cavity (hole) into the other polypeptide at an interface in which they interact. See e.g., WO 96/027011, WO 98/050431, U.S. Pat. No. 5,731,168, US2007/0178552, WO2009089004, US 20090182127. In particular, a combination of mutations in the CH3 domain can be used to preferentially form heterodimers, for example, S354C, T366W in the “knob” heavy chain, and Y349C, T366S, L368A, Y407V in the “hole” heavy chain. In some embodiments, the heterodimeric binuclease fusion protein disclosed herein includes a first CH3 domain having the knob mutation T366W and a second CH3 domain having the hole mutations T366S, L368A, and Y407V. (Numbering according to the EU index.)

In some embodiments, the CH3 mutations are those described by Zymeworks (US 2012/0149876 A1, incorporated herein by reference; and Von Kreudenstein, T. S. et al. mABs. 5 (2013), pp. 646-654) and include the following mutations: T350V, L351Y, F405A, and Y407V (first CH3 domain); and T350V, T366L, K392L, T394W (second CH3 domain). In some embodiments, the heterodimeric binuclease fusion protein disclosed herein includes a first CH3 domain having T350V, L351Y, F405A, and Y407V mutations and a second CH3 domain having T350V, T366L, K392L, T394W mutations. (Numbering according to the EU index.)

In some embodiments, the CH3 mutations are those described by Moore, G. L. et al (nABs, 3 (2011), pp. 546-557) and include the following mutations: S364H and F405A (first CH3 domain); and Y349T and T394F (second CH3 domain). In some embodiments, the heterodimeric binuclease fusion protein disclosed herein includes a first CH3 domain having S364H and F405A mutations and a second CH3 domain having Y349T and T394F mutations. (Numbering according to the EU index.)

In some embodiments, the CH3 mutations are those described by Gunasekaran, K. et al. (J. Biol. Chem., 285 (2010), pp, 19637-19646) and include the following mutations: K409D and K392D (first CH3 domain); and D399K and E365K (second CH3 domain). In some embodiments, the heterodimeric binuclease fusion protein disclosed herein includes a first CH3 domain having K409D and K392D mutations and a second CH3 domain having D399K and E365K mutations. (Numbering according to the EU index.)

The optimized binuclease fusion proteins of the disclosure may employ art-recognized Fc variants which are known to impart an alteration in effector function and/or FcR binding. For example, a change (e.g., a substitution) at one or more of the amino acid positions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351 A2, WO04/074455A2, WO04/099249A2, WO05/040217A2, WO04/044859, WO05/070963A1, WO05/077981A2, WO05/092925A2, WO05/123780A2, WO06/019447A1, WO06/047350A2, and WO06/085967A2; US Patent Publication Nos. US2007/0231329, US2007/0231329, US2007/0237765, US2007/0237766, US2007/0237767, US2007/0243188, US20070248603, US20070286859, US20080057056; or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; 7,083,784; and 7,317,091, each of which is incorporated by reference herein. In one embodiment, the specific change (e.g., the specific substitution of one or more amino acids disclosed in the art) may be made at one or more of the disclosed amino acid positions. In another embodiment, a different change at one or more of the disclosed amino acid positions (e.g., the different substitution of one or more amino acid position disclosed in the art) may be made.

Other amino acid mutations in the Fc domain are contemplated to reduce binding to the Fc gamma receptor and Fc gamma receptor subtypes. The assignment of amino acids residue numbers to an Fc domain is in accordance with the definitions of Kabat. See, e.g., Sequences of Proteins of Immunological Interest (Table of Contents, Introduction and Constant Region Sequences sections), 5th edition, Bethesda, MD:NIH vol. 1:647-723 (1991); Kabat et al., “Introduction” Sequences of Proteins of Immunological Interest, US Dept of Health and Human Services, NIH, 5th edition, Bethesda, MD vol. 1:xiii-xcvi (1991); Chothia & Lesk, J. Mol. Biol. 196:901-917 (1987); Chothia et al., Nature 342:878-883 (1989), each of which is herein incorporated by reference for all purposes.”

For example, mutations at positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 322, 324, 327, 329, 330, 331, 333, 334, 335, 337, 338, 340, 356, 360, 373, 376, 378, 379, 382, 388, 389, 398, 414, 416, 419, 430, 434, 435, 437, 438 or 439 of the Fc region can alter binding as described in U.S. Pat. No. 6,737,056, issued May 18, 2004, incorporated herein by reference in its entirety. This patent reported that changing Pro331 in IgG3 to Ser resulted in six fold lower affinity as compared to unmutated IgG3, indicating the involvement of Pro331 in Fc gamma RI binding. In addition, amino acid modifications at positions 234, 235, 236, and 237, 297, 318, 320 and 322 are disclosed as potentially altering receptor binding affinity in U.S. Pat. No. 5,624,821, issued Apr. 29, 1997 and incorporated herein by reference in its entirety. (Numbering according to the EU index.)

Further mutations contemplated for use include, e.g., those described in U.S. Pat. App. Pub. No. 2006/0235208, published Oct. 19, 2006 and incorporated herein by reference in its entirety. This publications describe Fc variants that exhibit reduced binding to Fc gamma receptors, reduced antibody dependent cell-mediated cytotoxicity, or reduced complement dependent cytotoxicity, that comprise at least one amino acid modification in the Fc region, including 232G, 234G, 234H, 235D, 235G, 235H, 236I, 236N, 236P, 236R, 237K, 237L, 237N, 237P, 238K, 239R, 265G, 267R, 269R, 270H, 297S, 299A, 299I, 299V, 325A, 325L, 327R, 328R, 329K, 330I, 330L, 330N, 330P, 330R, and 331L (numbering is according to the EU index), as well as double mutants 236R/237K, 236R/325L, 236R/328R, 237K/325L, 237K/328R, 325L/328R, 235G/236R, 267R/269R, 234G/235G, 236R/237K/325L, 236R/325L/328R, 235G/236R/237K, and 237K/325L/328R. Other mutations contemplated for use as described in this publication include 227G, 234D, 234E, 234G, 234I, 234Y, 235D, 235I, 235S, 236S, 239D, 246H, 255Y, 258H, 260H, 264I, 267D, 267E, 268D, 268E, 272H, 272I, 272R, 281D, 282G, 283H, 284E, 293R, 295E, 304T, 324G, 324I, 327D, 327A, 328A, 328D, 328E, 328F, 328I, 328M, 328N, 328Q, 328T, 328V, 328Y, 330I, 330L, 330Y, 332D, 332E, 335D, an insertion of G between positions 235 and 236, an insertion of A between positions 235 and 236, an insertion of S between positions 235 and 236, an insertion of T between positions 235 and 236, an insertion of N between positions 235 and 236, an insertion of D between positions 235 and 236, an insertion of V between positions 235 and 236, an insertion of L between positions 235 and 236, an insertion of G between positions 235 and 236, an insertion of A between positions 235 and 236, an insertion of S between positions 235 and 236, an insertion of T between positions 235 and 236, an insertion of N between positions 235 and 236, an insertion of D between positions 235 and 236, an insertion of V between positions 235 and 236, an insertion of L between positions 235 and 236, an insertion of G between positions 297 and 298, an insertion of A between positions 297 and 298, an insertion of S between positions 297 and 298, an insertion of D between positions 297 and 298, an insertion of G between positions 326 and 327, an insertion of A between positions 326 and 327, an insertion of T between positions 326 and 327, an insertion of D between positions 326 and 327, and an insertion of E between positions 326 and 327 (numbering is according to the EU index). Additionally, mutations described in U.S. Pat. App. Pub. No. 2006/0235208 include 227G/332E, 234D/332E, 234E/332E, 234Y/332E, 234I/332E, 234G/332E, 235I/332E, 235S/332E, 235D/332E, 235E/332E, 236S/332E, 236A/332E, 236S/332D, 236A/332D, 239D/268E, 246H/332E, 255Y/332E, 258H/332E, 260H/332E, 264I/332E, 267E/332E, 267D/332E, 268D/332D, 268E/332D, 268E/332E, 268D/332E, 268E/330Y, 268D/330Y, 272R/332E, 272H/332E, 283H/332E, 284E/332E, 293R/332E, 295E/332E, 304T/332E, 324I/332E, 324G/332E, 324I/332D, 324G/332D, 327D/332E, 328A/332E, 328T/332E, 328V/332E, 328I/332E, 328F/332E, 328Y/332E, 328M/332E, 328D/332E, 328E/332E, 328N/332E, 328Q/332E, 328A/332D, 328T/332D, 328V/332D, 328I/332D, 328F/332D, 328Y/332D, 328M/332D, 328D/332D, 328E/332D, 328N/332D, 328Q/332D, 330L/332E, 330Y/332E, 330I/332E, 332D/330Y, 335D/332E, 239D/332E, 239D/332E/330Y, 239D/332E/330L, 239D/332E/330I, 239D/332E/268E, 239D/332E/268D, 239D/332E/327D, 239D/332E/284E, 239D/268E/330Y, 239D/332E/268E/330Y, 239D/332E/327A, 239D/332E/268E/327A, 239D/332E/330Y/327A, 332E/330Y/268 E/327A, 239D/332E/268E/330Y/327A, Insert G>297-298/332E, Insert A>297-298/332E, Insert S>297-298/332E, Insert D>297-298/332E, Insert G>326-327/332E, Insert A>326-327/332E, Insert T>326-327/332E, Insert D>326-327/332E, Insert E>326-327/332E, Insert G>235-236/332E, Insert A>235-236/332E, Insert S>235-236/332E, Insert T>235-236/332E, Insert N>235-236/332E, Insert D>235-236/332E, Insert V>235-236/332E, Insert L>235-236/332E, Insert G>235-236/332D, Insert A>235-236/332D, Insert S>235-236/332D, Insert T>235-236/332D, Insert N>235-236/332D, Insert D>235-236/332D, Insert V>235-236/332D, and Insert L>235-236/332D (numbering according to the EU index) are contemplated for use. The mutant L234A/L235A is described, e.g., in U.S. Pat. App. Pub. No. 2003/0108548, published Jun. 12, 2003 and incorporated herein by reference in its entirety. In embodiments, the described modifications are included either individually or in combination. (Numbering according to the EU index.)

### Linker Domains

In some embodiments, an optimized binuclease fusion protein includes a linker domain. In some embodiments, an optimized binuclease fusion protein includes a plurality of linker domains. In some embodiments, the linker domain is a polypeptide linker. In certain aspects, it is desirable to employ a polypeptide linker to fuse Fc, or a variant or fragment thereof, with one or more nuclease domains to form an optimized binuclease fusion protein.

In one embodiment, the polypeptide linker is synthetic. As used herein, the term “synthetic” with respect to a polypeptide linker includes peptides (or polypeptides) which comprise an amino acid sequence (which may or may not be naturally occurring) that is linked in a linear sequence of amino acids to a sequence (which may or may not be naturally occurring) (e.g., a Fc sequence) to which it is not naturally linked in nature. For example, the polypeptide linker may comprise non-naturally occurring polypeptides which are modified forms of naturally occurring polypeptides (e.g., comprising a mutation such as an addition, substitution or deletion) or which comprise a first amino acid sequence (which may or may not be naturally occurring). The polypeptide linkers of the invention may be employed, for instance, to ensure that Fc, or a variant or fragment thereof, is juxtaposed to ensure proper folding and formation of a functional Fc, or a variant or fragment thereof. Preferably, a polypeptide linker compatible with the instant invention will be relatively non-immunogenic and not inhibit any non-covalent association among monomer subunits of a binding protein.

In certain embodiments, the optimized binuclease fusion protein employs an NLG linker as set forth in SEQ ID NO: 41.

In certain embodiments, the optimized binuclease fusion proteins of the disclosure employ a polypeptide linker to join any two or more domains in frame in a single polypeptide chain. In one embodiment, the two or more domains may be independently selected from any of the Fc domains, or variants or fragments thereof, or nuclease domains discussed herein. For example, in certain embodiments, a polypeptide linker can be used to fuse identical Fc fragments, thereby forming a homodimeric Fc region. In other embodiments, a polypeptide linker can be used to fuse different Fc fragments, thereby forming a heterodimeric Fc region. In other embodiments, a polypeptide linker of the invention can be used to genetically fuse the C-terminus of a first Fc fragment to the N-terminus of a second Fc fragment to form a complete Fc domain.

In one embodiment, a polypeptide linker comprises a portion of a Fc domain, or a variant or fragment thereof. For example, in one embodiment, a polypeptide linker can comprise a Fc fragment (e.g., C or N domain), or a different portion of a Fc domain or variant thereof.

In another embodiment, a polypeptide linker comprises or consists of a gly-ser linker. As used herein, the term “gly-ser linker” refers to a peptide that consists of glycine and serine residues. An exemplary gly/ser linker comprises an amino acid sequence of the formula (Gly₄Ser)n, wherein n is a positive integer (e.g., 1, 2, 3, 4, or 5). A preferred gly/ser linker is (Gly₄Ser)4. Another preferred gly/ser linker is (Gly₄Ser)3. Another preferred gly/ser linker is (Gly₄Ser)5. In certain embodiments, the gly-ser linker may be inserted between two other sequences of the polypeptide linker (e.g., any of the polypeptide linker sequences described herein). In other embodiments, a gly-ser linker is attached at one or both ends of another sequence of the polypeptide linker (e.g., any of the polypeptide linker sequences described herein). In yet other embodiments, two or more gly-ser linker are incorporated in series in a polypeptide linker.

In other embodiments, a polypeptide linker of the invention comprises a biologically relevant peptide sequence or a sequence portion thereof. For example, a biologically relevant peptide sequence may include, but is not limited to, sequences derived from an anti-rejection or anti-inflammatory peptide. Said anti-rejection or anti-inflammatory peptides may be selected from the group consisting of a cytokine inhibitory peptide, a cell adhesion inhibitory peptide, a thrombin inhibitory peptide, and a platelet inhibitory peptide. In a preferred embodiment, a polypeptide linker comprises a peptide sequence selected from the group consisting of an IL-1 inhibitory or antagonist peptide sequence, an erythropoietin (EPO)-mimetic peptide sequence, a thrombopoietin (TPO)-mimetic peptide sequence, G-CSF mimetic peptide sequence, a TNF-antagonist peptide sequence, an integrin-binding peptide sequence, a selectin antagonist peptide sequence, an anti-pathogenic peptide sequence, a vasoactive intestinal peptide (VIP) mimetic peptide sequence, a calmodulin antagonist peptide sequence, a mast cell antagonist, a SH3 antagonist peptide sequence, an urokinase receptor (UKR) antagonist peptide sequence, a somatostatin or cortistatin mimetic peptide sequence, and a macrophage and/or T-cell inhibiting peptide sequence. Exemplary peptide sequences, any one of which may be employed as a polypeptide linker, are disclosed in U.S. Pat. No. 6,660,843, which is incorporated by reference herein.

Other linkers that are suitable for use in optimized binuclease fusion proteins are known in the art, for example, the serine-rich linkers disclosed in U.S. Pat. No. 5,525,491, the helix forming peptide linkers (e.g., A(EAAAK)nA (n=2-5)) disclosed in Arai et al., Protein Eng 2001; 14:529-32, and the stable linkers disclosed in Chen et al., Mol Pharm 2011; 8:457-65, i.e., the dipeptide linker LE, a thrombin-sensitive disulfide cyclopeptide linker, and the alpha-helix forming linker LEA(EAAAK)₄ALEA(EAAAK)₄ALE (SEQ ID NO: 53).

Other exemplary linkers include GS linkers (i.e., (GS)n), GGSG (SEQ ID NO: 70) linkers (i.e., (GGSG)n), GSAT linkers (SEQ ID NO: 44), SEG linkers, and GGS linkers (i.e., (GGSGGS)n), wherein n is a positive integer (e.g., 1, 2, 3, 4, or 5). Other suitable linkers for use in the optimized binuclease fusion proteins can be found using publicly available databases, such as the Linker Database (ibi.vu.nl/programs/linkerdbwww). The Linker Database is a database of inter-domain linkers in multi-functional enzymes which serve as potential linkers in novel fusion proteins (see, e.g., George et al., Protein Engineering 2002; 15:871-9).

It will be understood that variant forms of these exemplary polypeptide linkers can be created by introducing one or more nucleotide substitutions, additions or deletions into the nucleotide sequence encoding a polypeptide linker such that one or more amino acid substitutions, additions or deletions are introduced into the polypeptide linker. Mutations may be introduced by standard techniques, such as site-directed mutagenesis and PCR-mediated mutagenesis.

Polypeptide linkers of the disclosure are at least one amino acid in length and can be of varying lengths. In one embodiment, a polypeptide linker of the invention is from about 1 to about 50 amino acids in length. As used in this context, the term “about” indicates +/−two amino acid residues. Since linker length must be a positive integer, the length of from about 1 to about 50 amino acids in length, means a length of from 1 to 48-52 amino acids in length. In another embodiment, a polypeptide linker of the disclosure is from about 10-20 amino acids in length. In another embodiment, a polypeptide linker of the disclosure is from about 15 to about 50 amino acids in length.

In another embodiment, a polypeptide linker of the disclosure is from about 20 to about 45 amino acids in length. In another embodiment, a polypeptide linker of the disclosure is from about 15 to about 25 amino acids in length. In another embodiment, a polypeptide linker of the disclosure is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 or more amino acids in length.

Polypeptide linkers can be introduced into polypeptide sequences using techniques known in the art. Modifications can be confirmed by DNA sequence analysis. Plasmid DNA can be used to transform host cells for stable production of the polypeptides produced.

### Exemplary Optimized Binuclease Fusion Proteins

The optimized binuclease fusion proteins of the invention are modular, and can be configured to incorporate various individual domains. For example, in one embodiment, the optimized binuclease fusion protein may include the mutant, human DNase1 A114F domain set forth in (SEQ ID NO: 21). In another embodiment, the optimized binuclease fusion protein may include the mutant, human DNase1 N18S/N106S/A114F domain set forth in SEQ ID NO: 24. In another embodiment, the optimized binuclease fusion protein may include the human, wild-type RNase1 domain set forth in SEQ ID NO: 27. In another embodiment, the optimized binuclease fusion protein may include the human, mutant RNase1 N34S/N76S/N88S domain set forth in SEQ ID NO: 28. In another embodiment, the optimized binuclease fusion protein may include the (Gly₄Ser)3 linker domain set forth in SEQ ID NO: 30. In another embodiment, the optimized binuclease fusion protein may include the NLG linker set forth in SEQ ID NO: 41. In another embodiment, the optimized binuclease fusion protein may include a VK3LP leader (SEQ ID NO: 54). It will be understood to the skilled artisan that these individual domains can be operably coupled to each other in any order to form an optimized binuclease fusion protein that is enzymatically active. For example, as detailed in the specific examples below, RNase1 can be operably coupled to an Fc domain. In another example, RNase1 can be operatively coupled to Fc domain via a (Gly₄Ser)3 linker domain. In yet another example, DNase1 A114F can be operatively coupled to Fc domain. In yet another example, DNase1 A114F can be operatively coupled to Fc domain via a (Gly₄Ser)3 linker domain. Various other configurations are possible, with non-limiting exemplary configurations disclosed herein, in FIG. 1 and in the Sequence Table.

In some embodiments, an optimized binuclease fusion protein comprises a wild-type, human RNase1 domain operably coupled to a mutant Fc domain comprising SCC hinge and CH2 mutations P238S and P331S, or fragment thereof, and a mutated human DNase1 domain operably coupled to the human RNase1, thereby forming a tandem homodimer. In some embodiments, the DNase1 is linked to the RNase1 via a peptide linker, such as an NLG linker disclosed herein. In some embodiments, the RNase1 is operably linked with or without a linker to the N-terminus of the Fc domain. In some embodiments, an optimized binuclease fusion protein comprises a polypeptide having the amino acid sequence set forth in SEQ ID NO: 1. In some embodiments, the RNase1 is operably linked with or without a linker to the C-terminus of the Fc domain. In some embodiments, an optimized binuclease fusion protein comprises a polypeptide having the amino acid sequence set forth in SEQ ID NO: 2. In some embodiments, the optimized binuclease fusion protein is homodimeric or heterodimeric.

In some embodiments, an optimized binuclease fusion protein is a heterodimer comprising mutant human DNase 1 domain operably coupled with or without a linker to a first mutant Fc domain, having SCC hinge, CH2 mutations P238S, P331S, and CH3 mutations T350V, L351Y, F405A and Y407V, or a variant or fragment thereof, and a wild-type human RNase 1 domain operably coupled with or without a linker to a second mutant Fc domain comprising SCC hinge, CH2 mutations P238S and P331S and CH3 mutations T350V, T366L, K392L and T394W, or a fragment thereof. In some embodiments, the DNase1 and RNase 1 are both linked to the N-terminus of their respective Fc domains. In some embodiments, an optimized binuclease fusion protein is a heterodimer comprising a polypeptide comprising the amino acid sequence set forth in SEQ ID NOs: 3 and a polypeptide comprising the amino acid sequence set for in SEQ ID NO: 4.

In some embodiments, an optimized binuclease fusion protein is a heterodimer comprising a mutant human DNase 1 domain and wild-type human RNase 1 domain, both operably coupled with or without a linker to a first mutant Fc domain, comprising SCC hinge, CH2 mutations P238S and P331S and CH3 mutations T350V, T366L, K392L and T394W, or a fragment thereof, and a second mutant Fc domain, having mutations T350V, T366L, K392L and T394W, or fragment thereof. In some embodiments, the DNase1 and RNase 1 are linked to the N-terminus and C-terminus, respectively, of the first and second Fc domains. In some embodiments, an optimized binuclease fusion protein is a heterodimer comprising a polypeptide comprising the sequence set forth in SEQ ID NO: 5 and a polypeptide comprising the amino acid sequence set forth in SEQ ID NO: 6.

In some embodiments, an optimized binuclease fusion protein is a heterodimer comprising a mutant human DNase 1 domain, operably coupled with or without a linker to a mutant Fc domain comprising SCC hinge, CH2 mutations P238S, P331S and CH3 mutations T350V, L351Y, F405A and Y407V, or fragment thereof, and wild-type human RNase 1 domain, operably coupled with or without a linker to a mutant Fc domain comprising SCC hinge, CH2 mutations P238S, P331S and CH3 mutations T350V, T366L, K392L and T394W, or fragment thereof. In some embodiments, the DNase1 is linked to the N-terminus of the Fc domain and the RNase1 is linked to the C-terminus of the Fc domain. In some embodiments, an optimized binuclease fusion protein is a heterodimer comprising a polypeptide comprising the sequence set forth in SEQ ID NO: 7 and a polypeptide comprising the amino acid sequence set forth in SEQ ID NO: 8.

In some embodiments, an optimized binuclease fusion protein is a heterodimer comprising a mutant human DNase 1 domain operably coupled with or without a linker to a mutant Fc domain comprising SCC hinge, CH2 mutations P238S, P331S and CH3 mutations T350V, L351Y, F405A and Y407V, or fragment thereof, and a wild-type human RNase 1 domain operably coupled with or without a linker to a mutant Fc domain comprising SCC hinge, CH2 mutations P238S, P331S and CH3 mutations T350V, T366L, K392L and T394W, or fragment thereof. In some embodiments, the DNase1 and RNase 1 are both linked to the C-terminus of their respective Fc domains. In some embodiments, an optimized binuclease fusion protein is a heterodimer comprising a polypeptide comprising the sequence set forth in SEQ ID NO: 9 and a polypeptide comprising the amino acid sequence set forth in SEQ ID NO: 10. In some embodiments, an optimized binuclease fusion protein is a heterodimer comprising a polypeptide comprising the sequence set forth in SEQ ID NO: 11 and a polypeptide comprising the amino acid sequence set forth in SEQ ID NO: 12.

In some embodiments, an optimized binuclease fusion protein is a heterodimer comprising a mutant human DNase 1 domain and wild-type human RNase 1 domain, both operably coupled with or without a linker to a mutant Fc domain comprising SCC hinge, CH2 mutations P238S, P331S and CH3 mutations T350V, L351Y, F405A and Y407V, or fragment thereof, and a mutant Fc domain comprising SCC hinge, CH2 mutations P238S, P331S and CH3 mutations T350V, T366L, K392L and T394W, or fragment thereof. In some embodiments, the DNase1 and RNase 1 are linked to the C-terminus and N-terminus, respectively, of the Fc domain. In some embodiments, an optimized binuclease fusion protein is a heterodimer comprising a polypeptide comprising the sequence set forth in SEQ ID NO:13 and a polypeptide comprising the amino acid sequence set forth in SEQ ID NO: 14.

In some embodiments, an optimized binuclease fusion protein is a heterodimer comprising a mutant human DNase 1 domain, operably coupled with or without a linker to a mutant Fc domain comprising SCC hinge, CH2 mutations P238S, P331S and CH3 mutations T350V, L351Y, F405A and Y407V, or fragment thereof, and wild-type human RNase 1 domain, operably coupled with or without a linker to a mutant Fc domain comprising SCC hinge, CH2 mutations P238S, P331S and CH3 mutations T350V, T366L, K392L and T394W, or fragment thereof. In some embodiments, the DNase1 is linked to the C-terminus of the Fc domain and the RNase1 is linked to the N-terminus of the Fc domain. In some embodiments, an optimized binuclease fusion protein is a heterodimer comprising a polypeptide comprising the sequence set forth in SEQ ID NO: 15 and a polypeptide comprising the amino acid sequence set forth in SEQ ID NO: 16.

In some embodiments, an optimized binuclease fusion protein comprising a polypeptide having an amino acid sequence at least 80% identical, such as 85%, 90%, 91%, 92%, 93%, 94%, 95%, 96%, 97%, 98%, 99%, or at least 99.5% identical to an amino acid sequence of any one of SEQ ID NOs: 1-17. In some embodiments, the polypeptide comprises an amino acid sequence set for in any one of SEQ ID NOs: 1-17.

In some embodiments, the foregoing optimized binuclease fusion proteins have a leader sequence.

It will be understood by one of ordinary skill that the leader and linker sequences are optional and are not limited to those described in the embodiments above. For example, the RNase and/or DNase domains can be directly fused to the N- and/or C-terminus of Fc, or variant or fragment thereof, the leader domain can be any of those known in the art to be useful for its intended purpose, e.g., to increase protein expression and/or secretion (e.g., a Gaussia luciferase signal peptide (MGVKVLFALICIAVAEA; SEQ ID NO: 31)); the linker can be any linker known in the art, e.g., (Gly₄Ser)n, NLG (VDGASSPVNVSSPSVQDI; SEQ ID NO: 41), LE, thrombin-sensitive disulphide cyclopeptide linker, LEA(EAAAK)₄ALEA(EAAAK)₄ (SEQ ID NO: 32), or an in vivo cleavable disulphide linker, as described herein. It will also be understood that it is within the abilities of a skilled artisan to make the corresponding changes to the amino acid sequences of the optimized binuclease fusion protein using routine cloning and recombination methods. It will also be understood that the asparagine residues in the nuclease domains (i.e., N34, N76, and N88 in RNase1, and N18 and N106 in DNase1) can be substituted with an amino acid other than serine (e.g., glutamine), as long as the amino acid does not serve as an acceptor for N-linked glycosylation.

### Methods of Making Optimized Binuclease Fusion Proteins

The optimized binuclease fusion proteins of this disclosure largely may be made in transformed or transfected host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used. The invention also includes a vector capable of expressing the peptides in an appropriate host. The vector comprises the DNA molecule that codes for the peptides operably coupled to appropriate expression control sequences. Methods of affecting this operative linking, either before or after the DNA molecule is inserted into the vector, are well known. Expression control sequences include promoters, activators, enhancers, operators, ribosomal nuclease domains, start signals, stop signals, cap signals, polyadenylation signals, and other signals involved with the control of transcription or translation.

The resulting vector having the DNA molecule thereon is used to transform or transfect an appropriate host. This transformation or transfection may be performed using methods well known in the art.

Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation or transfection, ease of recovery of the peptides, expression characteristics, bio-safety and costs. A balance of these factors must be struck with the understanding that not all hosts may be equally effective for the expression of a particular DNA sequence. Within these general guidelines, useful microbial hosts include bacteria (such as E. coli), yeast (such as Saccharomyces) and other fungi, insects, plants, mammalian (including human) cells in culture, or other hosts known in the art. In a preferred embodiment, the optimized binuclease fusion proteins are produced in CHO cells.

Next, the transformed or transfected host is cultured and purified. Host cells may be cultured under conventional fermentation or culture conditions so that the desired compounds are expressed. Such fermentation and culture conditions are well known in the art. Finally, the peptides are purified from culture by methods well known in the art.

The compounds may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al., Biochem Intl 1985; 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides since it is the most cost-effective method of making small peptides. Compounds that contain derivatized peptides or which contain non-peptide groups may be synthesized by well-known organic chemistry techniques.

Other methods are of molecule expression/synthesis are generally known in the art to one of ordinary skill.

Optimized Binuclease Fusion Proteins with Altered Glycosylation

Glycosylation (e.g., 0-lined or N-linked glycosylation) can impact the serum half-life of the optimized binuclease fusion proteins of the disclosure by, e.g., minimizing their removal from circulation by mannose and asialoglycoprotein receptors and other lectin-like receptors. Accordingly, in some embodiments, the optimized binuclease fusion proteins of the disclosure are prepared in aglycosylated, deglycosylated, or underglycosylated form. Preferably, N-linked glycosylation is altered and the optimized binuclease fusion protein is aglycosyated.

In some embodiments, all asparagine residues in an optimized binuclease fusion protein that conform to the Asn-X-Ser/Thr (X can be any other naturally occurring amino acid except Pro) consensus are mutated to residues that do not serve as acceptors of N-linked glycosylation (e.g., serine, glutamine), thereby eliminating glycosylation of the optimized binuclease fusion protein when synthesized in a cell that glycosylates proteins.

In some embodiments, optimized binuclease fusion proteins lacking N-linked glycosylation sites are produced in mammalian cells. In one embodiment, the mammalian cell is a CHO cell. Accordingly, in a specific embodiment, an aglycosylated optimized binuclease fusion protein is produced in a CHO cell.

In other embodiments, a reduction or lack of N-glycosylation is achieved by, e.g., producing optimized binuclease fusion proteins in a host (e.g., bacteria such as E. coli), mammalian cells engineered to lack one or more enzymes important for glycosylation, or mammalian cells treated with agents that prevent glycosylation, such as tunicamycin (an inhibitor of Dol-PP-GlcNAc formation).

In some embodiments, the optimized binuclease fusion proteins are produced in lower eukaryotes engineered to produce glycoproteins with complex N-glycans, rather than high mannose type sugars (see, e.g., US2007/0105127).

In some embodiments, glycosylated optimized binuclease fusion proteins (e.g., those produced in mammalian cells such as CHO cells) are treated chemically or enzymatically to remove one or more carbohydrate residues (e.g., one or more mannose, fucose, and/or N-acetylglucosamine residues) or to modify or mask one or more carbohydrate residues. Such modifications or masking may reduce binding of the optimized binuclease fusion proteins to mannose receptors, and/or asialoglycoprotein receptors, and/or other lectin-like receptors. Chemical deglycosylation can be achieved by treating an optimized binuclease fusion protein with trifluoromethane sulfonic acid (TFMS), as disclosed in, e.g., Sojar et al., JBC 1989; 264:2552-9 and Sojar et al., Methods Enzymol 1987; 138:341-50, or by treating with hydrogen fluoride, as disclosed in Sojar et al. (1987, supra). Enzymatic removal of N-linked carbohydrates from optimized binuclease fusion proteins can be achieved by treating an optimized binuclease fusion protein with protein N-glycosidase (PNGase) A or F, as disclosed in Thotakura et al. (Methods Enzymol 1987; 138:350-9). Other art-recognized commercially available deglycosylating enzymes that are suitable for use include endo-alpha-N-acetyl-galactosaminidase, endoglycosidase F1, endoglycosidase F2, endoglycosidase F3, and endoglycosidase H. In some embodiments, one or more of these enzymes can be used to deglycosylate the optimized binuclease fusion proteins of the disclosure. Alternative methods for deglycosylation are disclosed in, e.g., U.S. Pat. No. 8,198,063.

In some embodiments, the optimized binuclease fusion proteins are partially deglycosylated. Partial deglycosylation can be achieved by treating the optimized binuclease fusion proteins with an endoglycosidase (e.g., endoglycosidase H), which cleaves N-linked high mannose carbohydrate but not complex type carbohydrates, leaving a single GlcNAc residue linked to the asparagine. Optimized binuclease fusion proteins treated with endoglycosidase H will lack high mannose carbohydrates, resulting in a reduced interaction with the hepatic mannose receptor. Although this receptor recognizes terminal GlcNAc, the probability of a productive interaction with the single GlcNAc on the protein surface is not as great as with an intact high mannose structure.

In other embodiments, glycosylation of an optimized binuclease fusion protein is modified, e.g., by oxidation, reduction, dehydration, substitution, esterification, alkylation, sialylation, carbon-carbon bond cleavage, or the like, to reduce clearance of the optimized binuclease fusion proteins from blood. In some embodiments, the optimized binuclease fusion proteins are treated with periodate and sodium borohydride to modify the carbohydrate structure. Periodate treatment oxidizes vicinal diols, cleaving the carbon-carbon bond and replacing the hydroxyl groups with aldehyde groups; borohydride reduces the aldehydes to hydroxyls. Many sugar residues include vicinal diols and, therefore, are cleaved by this treatment. Prolonged serum half-life with periodate and sodium borohydride is exemplified by the sequential treatment of the lysosomal enzyme β-glucuronidase with these agents (see, e.g., Houba et al. (1996) Bioconjug Chem 1996:7:606-11; Stahl et al. PNAS 1976; 73:4045-9; Achord et al. Pediat. Res 1977; 11:816-22; Achord et al. Cell 1978; 15:269-78). A method for treatment with periodate and sodium borohydride is disclosed in Hickman et al., BBRC 1974; 57:55-61. A method for treatment with periodate and cyanoborohydride, which increases the serum half-life and tissue distribution of ricin, is disclosed in Thorpe et al. Eur J Biochem 1985; 147:197-206.

In one embodiment, the carbohydrate structures of an optimized binuclease fusion protein can be masked by addition of one or more additional moieties (e.g., carbohydrate groups, phosphate groups, alkyl groups, etc.) that interfere with recognition of the structure by a mannose or asialoglycoprotein receptor or other lectin-like receptors.

In some embodiments, one or more potential glycosylation sites are removed by mutation of the nucleic acid encoding the optimized binuclease fusion protein, thereby reducing glycosylation (underglycosylation) of the optimized binuclease fusion protein when synthesized in a cell that glycosylates proteins, e.g., a mammalian cell such as a CHO cell. In some embodiments, it may be desirable to selectively underglycosylate the nuclease domain of the optimized binuclease fusion proteins by mutating the potential N-linked glycosylation sites therein if, e.g., the underglycosylated optimized binuclease fusion protein exhibits increased activity or contributes to increased serum half-life. In other embodiments, it may be desirable to underglycosylate portions of theoptimized binuclease fusion protein such that regions other than the nuclease domain lack N-glycosylation if, for example, such a modification improves the serum half-life of the optimized binuclease fusion protein. Alternatively, other amino acids in the vicinity of glycosylation acceptors can be modified, disrupting a recognition motif for glycosylation enzymes without necessarily changing the amino acid that would normally be glycosylated.

In some embodiments, glycosylation of an optimized binuclease fusion protein can be altered by introducing glycosylation sites. For example, the amino acid sequence of the optimized binuclease fusion protein can be modified to introduce the consensus sequence for N-linked glycosylation of Asp-X-Ser/Thr (X is any amino acid other than proline). Additional N-linked glycosylation sites can be added anywhere throughout the amino acid sequence of the optimized binuclease fusion protein. Preferably, the glycosylation sites are introduced in position in the amino acid sequence that does not substantially reduce the nuclease (e.g., RNase and/or DNase) activity of the optimized binuclease fusion protein.

The addition of O-linked glycosylation sites has been reported to alter serum half-life of proteins, such as growth hormone, follicle-stimulating hormone, IGFBP-6, Factor IX, and many others (e.g., as disclosed in Okada et al., Endocr Rev 2011; 32:2-342; Weenen et al., J Clin Endocrinol Metab 2004; 89:5204-12; Marinaro et al., European Journal of Endocrinology 2000; 142:512-6; US 2011/0154516). Accordingly, in some embodiments, O-linked glycosylation (on serine/threonine residues) of the optimized binuclease fusion proteins is altered. Methods for altering O-linked glycosylation are routine in the art and can be achieved, e.g., by beta-elimination (see, e.g., Huang et al., Rapid Communications in Mass Spectrometry 2002; 16:1199-204; Conrad, Curr Protoc Mol Biol 2001; Chapter 17:Unit17.15A; Fukuda, Curr Protoc Mol Biol 2001; Chapter 17; Unit 17.15B; Zachara et al., Curr Protoc Mol Biol 2011; Unit 17.6;); by using commercially available kits (e.g., GlycoProfile™ Beta-Elimination Kit, Sigma); or by subjecting optimized binuclease fusion protein to treatment with a series of exoglycosidases such as, but not limited to, β1-4 galactosidase and β-N-acetylglucosaminidase, until only Gal β1-3GalNAc and/or GlcNAc β1-3GalNAc remains, followed by treatment with, e.g., endo-α-N-acetylgalactosaminidase (i.e., O-glycosidase). Such enzymes are commercially available from, e.g., New England Biolabs. In yet other embodiments, the optimized binuclease fusion proteins are altered to introduce O-linked glycosylation in the optimized binuclease fusion protein as disclosed in, e.g., Okada et al. (supra), Weenen et al. (supra), US2008/0274958; and US2011/0171218. In some embodiments, one or more O-linked glycosylation consensus sites are introduced into the optimized binuclease fusion protein, such as CXXGGT/S-C (SEQ ID NO: 33) (van den Steen et al., In Critical Reviews in Biochemistry and Molecular Biology, Michael Cox, ed., 1998; 33:151-208), NST-E/D-A (SEQ ID NO: 34), NITQS (SEQ ID NO: 35), QSTQS (SEQ ID NO: 36), D/E-FT-R/K-V (SEQ ID NO: 37), C-E/D-SN (SEQ ID NO: 38), and GGSC-K/R (SEQ ID NO: 39). Additional O-linked glycosylation sites can be added anywhere throughout the amino acid sequence of the optimized binuclease fusion protein. Preferably, the glycosylation sites are introduced in position in the amino acid sequence that does not substantially reduce the nuclease (e.g., RNase and/or DNase) activity of the optimized binuclease fusion protein. Alternatively, O-linked sugar moieties are introduced by chemically modifying an amino acid in the optimized binuclease fusion protein as described in, e.g., WO 87/05330 and Aplin et al., CRC Crit Rev Biochem 1981; 259-306).

In some embodiments, both N-linked and O-linked glycosylation sites are introduced into the optimized binuclease fusion proteins, preferably in positions in the amino acid sequence that do not substantially reduce the nuclease (e.g., RNase and/or DNase) activity of the optimized binuclease fusion protein.

It is well within the abilities of the skilled artisan to introduce, reduce, or eliminate glycosylation (e.g., N-linked or O-linked glycosylation) in an optimized binuclease fusion protein and determine using routine methods in the art whether such modifications in glycosylation status increases or decreases the nuclease activity or serum half-life of the optimized binuclease fusion protein.

In some embodiments, the optimized binuclease fusion protein may comprise an altered glycoform (e.g., an underfucosylated or fucose-free glycan).

In some embodiments, an optimized binuclease fusion protein with altered glycosylation has a serum half-life that is increased at least about 1.5-fold, such as at least 3-fold, at least 5-fold, at least 10-fold, at least about 20-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1000-fold, or 1000-fold or greater relative to the corresponding glycosylated optimized binuclease fusion proteins (e.g., an optimized binuclease fusion protein in which potential N-linked glycosylation sites are not mutated). Routine art-recognized methods can be used to determine the serum half-life of optimized binuclease fusion proteins with altered glycosylation status.

In some embodiments, an optimized binuclease fusion protein with altered glycosylation (e.g., a aglycosylated, deglycosylated, or underglycosylated optimized binuclease fusion proteins) retains at least 50%, such as at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or 100% of the activity of the corresponding glycosylated optimized binuclease fusion protein (e.g., an optimized binuclease fusion protein in which potential N-linked glycosylation sites are not mutated).

In some embodiments, altering the glycosylation status of the optimized binuclease fusion proteins may increase nuclease activity, either by directly increasing enzymatic activity, or by increasing bioavailability (e.g., serum half-life). Accordingly, in some embodiments, the nuclease activity of an optimized binuclease fusion protein with altered glycosylation is increased by at least 1.3-fold, such as at least 1.5-fold, at least 2-fold, at least 2.5-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5 fold, or 10-fold or greater, relative to the corresponding glycosylated optimized binuclease fusion protein (e.g., an optimized binuclease fusion protein in which potential N-linked glycosylation sites are not mutated).

The skilled artisan can readily determine the glycosylation status of optimized binuclease fusion proteins using art-recognized methods. In a preferred embodiment, the glycosylation status is determined using mass spectrometry. In other embodiments, interactions with Concanavalin A (Con A) can be assessed to determine whether an optimized binuclease fusion protein is underglycosylated. An underglycosylated optimized binuclease fusion protein is expected to exhibit reduced binding to Con A-Sepharose when compared to the corresponding glycosylated optimized binuclease fusion protein. SDS-PAGE analysis can also be used to compare the mobility of an underglycosylated protein and corresponding glycosylated protein. The underglycosylated protein is expected to have a greater mobility in SDS-PAGE compared to the glycosylated protein. Other suitable art-recognized methods for analyzing protein glycosylation status are disclosed in, e.g., Roth et al., International Journal of Carbohydrate Chemistry 2012; 1-10.

Pharmacokinetics, such as serum half-life, of optimized binuclease fusion proteins with different glycosylation status can be assayed using routine methods, e.g., by introducing the optimized binuclease fusion proteins in mice, e.g., intravenously, taking blood samples at pre-determined time points, and assaying and comparing levels and/or enzymatic activity of the optimized binuclease fusion proteins in the samples.

### Pharmaceutical Compositions

In certain embodiments, an optimized binuclease fusion protein is administered alone. In certain embodiments, an optimized binuclease fusion protein is administered prior to the administration of at least one other therapeutic agent. In certain embodiments, an optimized binuclease fusion protein is administered concurrent with the administration of at least one other therapeutic agent. In certain embodiments, an optimized binuclease fusion protein is administered subsequent to the administration of at least one other therapeutic agent. In other embodiments, an optimized binuclease fusion protein is administered prior to the administration of at least one other therapeutic agent. As will be appreciated by one of skill in the art, in some embodiments, the optimized binuclease fusion protein is combined with the other agent/compound. In some embodiments, the optimized binuclease fusion protein and other agent are administered concurrently. In some embodiments, the optimized binuclease fusion protein and other agent are not administered simultaneously, with the optimized binuclease fusion protein being administered before or after the agent is administered. In some embodiments, the subject receives both the optimized binuclease fusion protein and the other agent during a same period of prevention, occurrence of a disorder, and/or period of treatment.

Pharmaceutical compositions of the invention can be administered in combination therapy, i.e., combined with other agents. In certain embodiments, the combination therapy comprises the optimized binuclease fusion protein, in combination with at least one other agent. Agents include, but are not limited to, in vitro synthetically prepared chemical compositions, antibodies, antigen binding regions, and combinations and conjugates thereof. In certain embodiments, an agent can act as an agonist, antagonist, allosteric modulator, or toxin.

In certain embodiments, the invention provides for pharmaceutical compositions comprising a optimized binuclease fusion protein together with a pharmaceutically acceptable diluent, carrier, solubilizer, emulsifier, preservative and/or adjuvant.

In certain embodiments, the invention provides for pharmaceutical compositions comprising a optimized binuclease fusion protein and a therapeutically effective amount of at least one additional therapeutic agent, together with a pharmaceutically acceptable diluent, carrier, solubilizer, emulsifier, preservative and/or adjuvant.

In certain embodiments, acceptable formulation materials preferably are nontoxic to recipients at the dosages and concentrations employed. In some embodiments, the formulation material(s) are for s.c. and/or I.V. administration. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as gelatin); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In some embodiments, the formulation comprises PBS; 20 mM NaOAC, pH 5.2, 50 mM NaCl; and/or 10 mM NAOAC, pH 5.2, 9% Sucrose.

In certain embodiments, a optimized binuclease fusion protein and/or a therapeutic molecule is linked to a half-life extending vehicle known in the art. Such vehicles include, but are not limited to, polyethylene glycol, glycogen (e.g., glycosylation of the optimized binuclease fusion protein), and dextran. Such vehicles are described, e.g., in U.S. application Ser. No. 09/428,082, now U.S. Pat. No. 6,660,843 and published PCT Application No. WO 99/25044.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibodies of the invention.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In some embodiments, the saline comprises isotonic phosphate-buffered saline. In certain embodiments, pharmaceutical compositions comprise Tris buffer of about pH 7.0-8.5, or acetate buffer of about H 4.0-5.5, which can further include sorbitol or a suitable substitute therefore. In certain embodiments, a composition comprising an optimized binuclease fusion protein, with or without at least one additional therapeutic agents, can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising an optimized binuclease fusion protein, with or without at least one additional therapeutic agent, can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising a desired optimized binuclease fusion protein, with or without additional therapeutic agents, in a pharmaceutically acceptable vehicle. In certain embodiments, a vehicle for parenteral injection is sterile distilled water in which an optimized binuclease fusion protein, with or without at least one additional therapeutic agent, is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, an optimized binuclease fusion protein, with or without at least one additional therapeutic agent, can be formulated as a dry powder for inhalation. In certain embodiments, an inhalation solution comprising an optimized binuclease fusion protein, with or without at least one additional therapeutic agent, can be formulated with a propellant for aerosol delivery. In certain embodiments, solutions can be nebulized. Pulmonary administration is further described in PCT application no. PCT/US94/001875, which describes pulmonary delivery of chemically modified proteins.

In certain embodiments, it is contemplated that formulations can be administered orally. In certain embodiments, an optimized binuclease fusion protein, with or without at least one additional therapeutic agents, that is administered in this fashion can be formulated with or without those carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of an optimized binuclease fusion protein and/or any additional therapeutic agents. In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of an optimized binuclease fusion protein, with or without at least one additional therapeutic agents, in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving an optimized binuclease fusion protein, with or without at least one additional therapeutic agent(s), in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al, Biopolymers, 22:547-556 (1983)), poly (2-hydroxyethyl-methacrylate) (Langer et al., J Biomed Mater Res, 15: 167-277 (1981) and Langer, Chem Tech, 12:98-105 (1982)), ethylene vinyl acetate (Langer et al, supra) or poly-D(−)-3-hydroxybutyric acid (EP 133,988). In certain embodiments, sustained release compositions can also include liposomes, which can be prepared by any of several methods known in the art. See, e.g., Eppstein et al, PNAS, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In certain embodiments, the effective amount of a pharmaceutical composition comprising an optimized binuclease fusion protein, with or without at least one additional therapeutic agent, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which an optimized binuclease fusion protein, with or without at least one additional therapeutic agent, is being used, the route of administration, and the size (body weight, body surface or organ size) and/or condition (the age and general health) of the patient. In certain embodiments, the clinician can titer the dosage and modify the route of administration to obtain the optimal therapeutic effect. In certain embodiments, a typical dosage can range from about 0.1 μg/kg to up to about 100 mg/kg or more, depending on the factors mentioned above. In certain embodiments, the dosage can range from 0.1 μg/kg up to about 100 mg/kg; or 1 μg/kg up to about 100 mg/kg; or 5 μg/kg up to about 100 mg/kg.

In certain embodiments, the frequency of dosing will take into account the pharmacokinetic parameters of an optimized binuclease fusion protein and/or any additional therapeutic agents in the formulation used. In certain embodiments, a clinician will administer the composition until a dosage is reached that achieves the desired effect. In certain embodiments, the composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device.

In certain embodiments, the composition can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

In certain embodiments, it can be desirable to use a pharmaceutical composition comprising an optimized binuclease fusion protein, with or without at least one additional therapeutic agent, in an ex vivo manner. In such instances, cells, tissues and/or organs that have been removed from the patient are exposed to a pharmaceutical composition comprising an optimized binuclease fusion protein, with or without at least one additional therapeutic agent, after which the cells, tissues and/or organs are subsequently implanted back into the patient.

In certain embodiments, an optimized binuclease fusion protein and/or any additional therapeutic agents can be delivered by implanting certain cells that have been genetically engineered, using methods such as those described herein, to express and secrete the polypeptides. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### In Vitro Assays

Various in vitro assays known in the art can be used to assess the efficacy of the optimized binuclease fusion proteins of the invention.

For example, cultured human PBMCs from normal or lupus patient PBMCs are isolated, cultured, and treated with various stimuli (e.g., TLR ligands, costimulatory antibodies, immune complexes, and normal or autoimmune sera), in the presence or absence of the optimized binuclease fusion proteins. Cytokine production by the stimulated cells can be measured using commercially available reagents, such as the antibody pair kits from Biolegend (San Diego, CA) for various cytokines (e.g., IL-6, IL-8, IL-10, IL-4, IFN-gamma, and TNF-alpha). Culture supernatants are harvested at various time points as appropriate for the assay (e.g., 24, 48 hours, or later time points) to determine the effects that the optimized binuclease fusion proteins have on cytokine production. IFN-alpha production is measured using, e.g., anti-human IFN-alpha antibodies and standard curve reagents available from PBL interferon source (Piscataway, NJ). Similar assays are performed using human lymphocyte subpopulations (isolated monocytes, B cells, pDCs, T cells, etc.); purified using, e.g., commercially available magnetic bead based isolation kits available from Miltenyi Biotech (Auburn, CA).

Multi-color flow cytometry can be used to assess the effects of the optimized binuclease fusion proteins on immune cell activation by measuring the expression of lymphocyte activation receptors such as CD5, CD23, CD69, CD80, CD86, and CD25 in PBMCs or isolated cell subpopulations at various time points after stimulation using routine art-recognized methods.

The efficacy of optimized binuclease fusion proteins can also be tested by incubating SLE patient serum with normal human pDCs to activate IFN output, as described in, e.g., Ahlin et al., Lupus 2012:21:586-95; Mathsson et al., Clin Expt Immunol 2007; 147:513-20; and Chiang et al., J Immunol 2011; 186:1279-1288. Without being bound by theory, circulating nucleic acid-containing immune complexes in SLE patient sera facilitate nucleic acid antigen entry into pDC endosomes via Fc receptor-mediated endocytosis, followed by binding of nucleic acids to and activation of endosomal TLRs 7, 8, and 9. To assess the impact of the optimized binuclease fusion proteinss, SLE patient sera or plasma is pretreated with the optimized binuclease fusion proteins, followed by addition to cultures of pDC cells isolated from healthy volunteers. Levels of IFN-α produced are then determined at multiple time points. By degrading nucleic-acid containing immune complexes, effective optimized binuclease fusion proteins are expected to reduce the quantity of IFN-α produced.

The effectiveness of optimized binuclease fusion proteins is demonstrated by comparing the results of an assay from cells treated with an optimized binuclease fusion protein disclosed herein to the results of the assay from cells treated with control formulations. After treatment, the levels of the various markers (e.g., cytokines, cell-surface receptors, proliferation) described above are generally improved in an effective optimized binuclease fusion protein treated group relative to the marker levels existing prior to the treatment, or relative to the levels measured in a control group.

### Methods of Treatment

The optimized binuclease fusion proteins of the disclosure are particularly effective in the treatment of autoimmune disorders or abnormal immune responses. In this regard, it will be appreciated that the optimized binuclease fusion proteins of the present disclosure may be used to control, suppress, modulate, treat, or eliminate unwanted immune responses to both external and autoantigens.

In another aspect, an optimized binuclease fusion protein is adapted for preventing (prophylactic) or treating (therapeutic) a disease or disorder, such as an autoimmune disease, in a mammal by administering an optimized binuclease fusion protein in a therapeutically effective amount or a sufficient amount to the mammal in need thereof, wherein the disease is prevented or treated. Any route of administration suitable for achieving the desired effect is contemplated by the invention (e.g., intravenous, intramuscular, subcutaneous). Treatment of the disease condition may result in a decrease in the symptoms associated with the condition, which may be long-term or short-term, or even a transient beneficial effect.

Numerous disease conditions are suitable for treatment with optimized binuclease fusion proteins of the disclosure. For example, in some aspects, the disease or disorder is an autoimmune disease or cancer. In some such aspects, the autoimmune disease is insulin-dependent diabetes mellitus, multiple sclerosis, experimental autoimmune encephalomyelitis, rheumatoid arthritis, experimental autoimmune arthritis, myasthenia gravis, thyroiditis, an experimental form of uveoretinitis, Hashimoto's thyroiditis, primary myxoedema, thyrotoxicosis, pernicious anaemia, autoimmune atrophic gastritis, Addison's disease, premature menopause, male infertility, juvenile diabetes, Goodpasture's syndrome, pemphigus vulgaris, pemphigoid, sympathetic ophthalmia, phacogenic uveitis, autoimmune haemolytic anaemia, idiopathic leucopenia, primary biliary cirrhosis, active chronic hepatitis Hbs-ve, cryptogenic cirrhosis, ulcerative colitis, Sjogren's syndrome, scleroderma, Wegener's granulomatosis, polymyositis, dermatomyositis, discoid LE, SLE, or connective tissue disease.

In a specific embodiment, an optimized binuclease fusion protein is used to prevent or treat SLE or Sjogren's syndrome. The effectiveness of an optimized binuclease fusion protein is demonstrated by comparing the IFN-alpha levels, IFN-alpha response gene levels, autoantibody titers, kidney function and pathology, and/or circulating immune complex levels in mammals treated with an optimized binuclease fusion protein disclosed herein to mammals treated with control formulations.

For example, a human subject in need of treatment is selected or identified (e.g., a patient who fulfills the American College of Rheumatology criteria for SLE, or a patient who fulfills the American-European Consensus Sjogren's Classification Criteria). The subject can be in need of, e.g., reducing a cause or symptom of SLE or Sjogren's syndrome. The identification of the subject can occur in a clinical setting, or elsewhere, e.g., in the subject's home through the subject's own use of a self-testing kit.

At time zero, a suitable first dose of an optimized binuclease fusion protein is administered to the subject. The optimized binuclease fusion protein is formulated as described herein. After a period of time following the first dose, e.g., 7 days, 14 days, and 21 days, the subject's condition is evaluated, e.g., by measuring IFN-alpha levels, IFN-alpha response gene levels, autoantibody titers, kidney function and pathology, and/or circulating immune complex levels. Other relevant criteria can also be measured. The number and strength of doses are adjusted according to the subject's needs. After treatment, the subject's IFN-alpha levels, IFN-alpha response gene levels, autoantibody titers, kidney function and pathology, and/or circulating immune complex levels are lowered and/or improved relative to the levels existing prior to the treatment, or relative to the levels measured in a similarly afflicted but untreated/control subject.

In another example, a rodent subject in need of treatment is selected or identified (see, e.g., Example 7). The identification of the subject can occur in a laboratory setting or elsewhere. At time zero, a suitable first dose of an optimized binuclease fusion protein is administered to the subject. The optimized binuclease fusion protein is formulated as described herein. After a period of time following the first dose, e.g., 7 days, 14 days, and 21 days, the subject's condition is evaluated, e.g., by measuring IFN-alpha levels, IFN-alpha response gene levels, autoantibody titers, kidney function and pathology, and/or circulating immune complex levels. Other relevant criteria can also be measured. The number and strength of doses are adjusted according to the subject's needs.

After treatment, the subject's IFN-alpha levels, IFN-alpha response gene levels, autoantibody titers, kidney function and pathology, and/or circulating immune complex levels are lowered and/or improved relative to the levels existing prior to the treatment, or relative to the levels measured in a similarly afflicted but untreated/control subject.

Another aspect of the present invention is to use gene therapy methods for treating or preventing disorders, diseases, and conditions with one or more optimized binuclease fusion proteins. The gene therapy methods relate to the introduction of optimized binuclease fusion protein nucleic acid (DNA, RNA and antisense DNA or RNA) sequences into an animal in need thereof to achieve expression of the polypeptide or polypeptides of the present disclosure. This method can include introduction of one or more polynucleotides encoding an optimized binuclease fusion protein of the present disclosure operably coupled to a promoter and any other genetic elements necessary for the expression of the polypeptide by the target tissue.

In gene therapy applications, optimized binuclease fusion protein genes are introduced into cells in order to achieve in vivo synthesis of a therapeutically effective genetic product. “Gene therapy” includes both conventional gene therapies where a lasting effect is achieved by a single treatment, and the administration of gene therapeutic agents, which involves the one time or repeated administration of a therapeutically effective DNA or mRNA. The oligonucleotides can be modified to enhance their uptake, e.g., by substituting their negatively charged phosphodiester groups by uncharged groups.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al, Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1

### Generating Optimized Binuclease Fusion Protein Encoding Expression Vectors

Various embodiments of the optimized binuclease fusion proteins of the disclosure are shown in FIG. 1, with amino acid sequences of each presented in the Sequence Table. As exemplary optimized binuclease fusion proteins, binuclease fusion proteins with the configurations shown in FIG. 1 were constructed. Specifically, starting from the amino acid sequence of the optimized binuclease fusion proteins, polynucleotides encoding the optimized binuclease fusion proteins were directly synthesized using codon optimization by Genescript (Genescript, Piscatawy, N.J.) to allow for optimal expression in mammalian cells. The process of optimization involved, e.g., avoiding regions of very high (>80%) or very low (<30%) GC content when possible, and avoiding cis-acting sequence motifs, such as internal TATA-boxes, chi-sites and ribosomal entry sites, AT-rich or GC-rich sequence stretches, RNA instability motifs, repeat sequences and RNA secondary structures, and cryptic splice donor and acceptor sites in higher eukaryotes. DNAs encoding the optimized binuclease fusion proteins are cloned into the pcDNA3.1+ mammalian expression vector. Optimized binuclease fusion proteins with the following configurations were generated.

Tandem homodimer RSLV-145 (SEQ ID NO: 1) has the configuration DNase-linker-RNase-Fc, wherein a wild-type, human RNase1 domain (SEQ ID NO: 27) is operably coupled without a linker to the N-terminus to a mutant Fc region comprising SCC hinge and CH2 mutations P238S, P331S (SEQ ID NO: 55) and a mutant human DNase1 domain (SEQ ID NO: 25) is operably coupled to the N-terminus of the RNase1 domain via a NLG linker (SEQ ID NO: 41).

To preferentially form heterodimers, each of the Fc domains in the following constructs included complementary CH3 mutations: T350V, L351Y, F405A, and Y407V; and T350V, T366L, K392L, and T394W (numbering according to the EU index.)

Tandem heterodimer RSLV-147 has the configuration DNase-Fc (SEQ ID NO: 3) and RNase-Fc (SEQ ID NO: 4), wherein a mutant human DNase1 domain (SEQ ID NO: 25) is operably coupled to the N-terminus of a first mutant Fc region comprising SCC hinge, CH2 mutations P238S, P331S and CH3 mutations, and wherein a wild-type, human RNase1 domain (SEQ ID NO: 27) is operably coupled to the N-terminus of a second mutant Fc region comprising SCC hinge, CH2 mutations P238S, P331S and CH3 mutations.

Heterodimer RSLV-148 has the configuration DNase-first Fc domain-linker-RNase (SEQ ID NO: 5) and a mutant second Fc domain (N-terminal truncation including first cysteine in CCC hinge) comprising CH2 mutations P238S, P331S and CH3 mutations (SEQ ID NO: 6), wherein a mutant human DNase1 domain (SEQ ID NO: 25) is operably coupled to the N-terminus of a first mutant Fc region comprising SCC hinge, CH2 mutations P238S, P331S and CH3 mutations, and wherein a wild-type, human RNase1 domain (SEQ ID NO: 27) is operably coupled via an NLG linker to the C-terminus of the first Fc region.

Heterodimer RSLV-149 has the configuration DNase-Fc (SEQ ID NO: 7) and Fc-linker-RNase (SEQ ID NO: 8), wherein a mutant human DNase1 domain (SEQ ID NO: 25) is operably coupled to the N-terminus of a first mutant Fc region comprising SCC hinge, CH2 mutations P238S, P331S and CH3 mutations, and wherein a wild-type, human RNase1 domain (SEQ ID NO: 27) is operably coupled via an NLG linker to the C-terminus of a second mutant Fc region comprising CH2 mutations P238S, P331S and CH3 mutations (SEQ ID NO:6).

Heterodimer RSLV-152 has the configuration RNase-first mutant Fc-linker-DNase (SEQ ID NO: 13) and a second mutant Fc domain comprising CH2 mutations P238S, P331S, and CH3 mutations (SEQ ID NO: 14), wherein a wild-type, human RNase1 domain (SEQ ID NO: 27) is operably coupled to the N-terminus of a first mutant Fc region comprising SCC hinge and CH2 mutations P238S, P331S and CH3 mutations, and wherein a mutant human DNase1 domain (SEQ ID NO: 25) is operably coupled via an NLG liner to the C-terminus of the first mutant Fc region.

Heterodimer RSLV-153 has the configuration Fc-linker-DNase (SEQ ID NO: 15) and RNase-Fc (SEQ ID NO: 16), wherein a mutant human DNase1 domain (SEQ ID NO: 25) is operably coupled via an NLG linker to the C-terminus of a first mutant Fc region comprising CH2 mutations P238S, P331S and CH3 mutations, and wherein a wild-type, human RNase1 domain (SEQ ID NO: 27) is operably coupled to the N-terminus of a second mutant Fc region comprising SCC hinge, CH2 mutations P238S, P331S and CH3 mutations.

Constructs RLSV-327 (a binuclease containing RNase 1 and DNase 1 linked to human serum albumin; RNase-linker-HSA-linker-DNase E13R/N74K/A114F/T205K) and RSLV-132 (RNase-Fc), containing DNase and RNase moieties, were used as controls.

### Example 2

### Transient Expression of and Stable Mammalian Cell Lines Expressing Optimized Binuclease Fusion Proteins

For transient expression, expression vectors from Example 1 containing the optimized binuclease fusion protein inserts are transiently trasfected using FreeStyle™ MAX Reagent into Chinese Hamster Ovary (CHO) cells, e.g., CHO-S cells (e.g., FreeStyle™ CHO-S cells, Invitrogen), using the manufacturer recommended transfection protocol. CHO-S cells are maintained in FreeStyle™ CHO Expression Medium containing 2 mM L-Glutamine and penicillin-streptomycin.

Stable CHO-S cell lines expressing the optimized binuclease fusion proteins are generated using routine methods known in the art. For example, CHO-S cells can be infected with a virus (e.g., retrovirus, lentivirus) comprising the nucleic acid sequences of an optimized binuclease fusion protein, as well as the nucleic acid sequences encoding a marker (e.g., GFP, surface markers selectable by magnetic beads) that is selected for using, e.g., flow cytometry or magnetic bead separation (e.g., MACSelect™ system). Alternatively, CHO-S cells are transfected using any transfection method known in the art, such as electroporation (Lonza) or the FreeStyle™ MAX Reagent as mentioned above, with a vector comprising the nucleic acid sequences of the optimized binuclease fusion proteins and a selectable marker, followed by selection using, e.g., flow cytometry. The selectable marker can be incorporated into the same vector as that encoding the optimized binuclease fusion proteins or a separate vector.

Optimized binuclease fusion proteins are purified from culture supernatant by capturing the molecules using a column packed with Protein-A sepharose beads, followed by washes in column wash buffer (e.g., 90 mM Tris, 150 mM NaCl, 0.05% sodium azide) and releasing the molecules from the column using a suitable elution buffer (e.g., 0.1 M citrate buffer, pH 3.0). The eluted material is further concentrated by buffer exchange through serial spins in PBS using Centricon concentrators, followed by filtration through 0.2 m filter devices. The concentration of the optimized binuclease fusion proteins is determined using standard spectrophotometric methods (e.g., Bradford, BCA, Lowry, Biuret assays).

### Example 3

### Nuclease Activity of Purified Optimized Binuclease Fusion Proteins

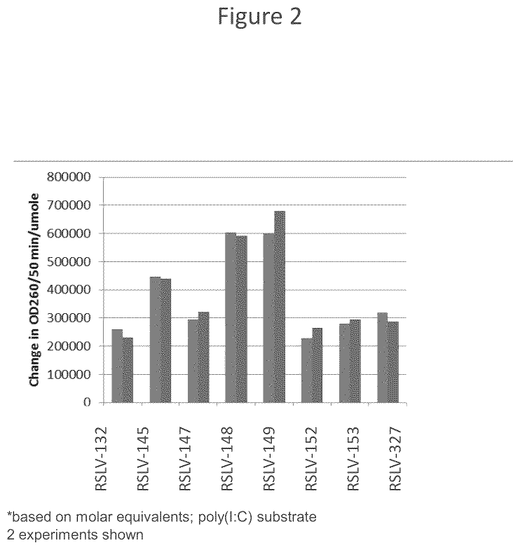

RNase activity of optimized binuclease fusion proteins present in mouse sera was analyzed. The proteins were added at doses of 12.5 to 100 ng to 2.5 mg/ml of poly-IC (Sigma) in 50 mM Hepes and 100 mM NaCl at pH 7.3 and incubated for 37° C. for 50 minutes. TCA was added to final concentration of 5% and left on ice. Samples were filtered to remove precipated and the filtrate was collected for OD₂₆₀ readings. The results are shown in FIG. 2. RSLV-132 and RSLV-145, both containing 2 RNase moieties per molar equivalent of construct, were both active, with RSLV-145 being more active than RSLV-132. The other constructs (RSLV-147, RSLV-152, RSLV-153 and RSLV-327), containing only one RNase moiety on a molar basis, were comparable and in line with RSLV-132. Surprisingly, RSLV-148 and RLSV-149 possessed activity greater than RSLV-132 and other single RNase constructs. These constructs each contain the RNase moiety attached at the C-terminus of the Fc domain.

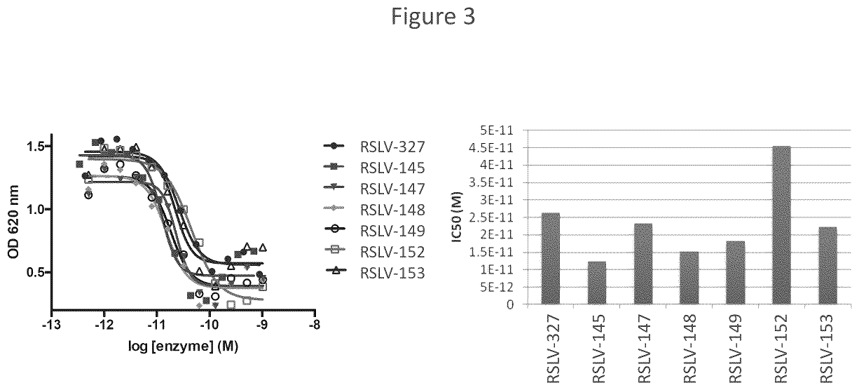

DNase1 activity of the optimized binuclease fusion proteins containing DNase1 domains was measured using ODN-2006-G5 (InvivoGen, tlrl-2006g5), a DNA oligonucleotide agonist of TLR9. The proteins, in dosages ranging from 0.24 ng/ml to 500 ng/ml) were incubated for 1 hour at 37° C. with ODN-2006-G5 in DMEM containing 25 mM Hepes and 10% FBS. The reaction mixtures were applied to hTLR9 HEKBlue cells, engineered to secrete alkaline phosphatase (SEAP) in response to TLR9 agonists, overnight at 37° C. The culture media was harvested and assayed for SEAP using a colormetric substrate and then read at OD₆₂₀. IC50 values were calculated using GraphPad Prism® version 6.0e software. Results are shown in FIG. 3. All 6 constructs (RSLV-145, RSLV-147, RSLV-148, RSLV-149, RSLV-152 and RSLV-153) possessed robust DNase activity and were at least 5000-fold more active than recombinant huDNase1. RSLV-145 appeared to be approximately 2× more active than other heterodimer constructs and RSLV-327, likely due to the 2 DNase domains in RSLV-145 compared to 1 DNase domain in the other constructs. RSLV-152 was consistently the least active of the constructs.

### Example 4

### Efficacy of Optimized Binuclease Fusion Proteins In Vitro

### Effects of Optimized Binuclease Fusion Proteins on Cytokine Expression

Human PBMCs are isolated from normal patients and lupus patients and cultured. The cells are treated with various stimulatory TLR ligands, costimulatory antibodies, immune complexes, and normal or autoimmune sera, with or without the optimized binuclease fusion proteins of Example 2. Culture supernatant is collected at various time points (e.g., 6 hrs, 12 hrs, 24 hrs, 48 hrs, etc) and levels of a panel of cytokines, including human IL-6, IL-8, IL-10, IL-4, IFN-gamma, IFN-alpha and TNF-alpha are measured using commercially available ELISA kits from, e.g., Thermo Fisher Scientific, Inc. Effective optimized binuclease fusion proteins are expected to reduce the levels of cytokines produced by stimulated PBMCs relative to controls.

### Effects of Optimized Binuclease Fusion Proteins on Lymphocyte Activation Receptor Expression

Human PBMCs are isolated from normal patients and lupus patients and cultured. The cells are treated with various stimulatory TLR ligands, costimulatory antibodies, immune complexes, and normal or autoimmune sera, with or without the optimized binuclease fusion proteins of Example 2. Cells are then subjected to multi-color flow cytometry to measure the expression of lymphocyte activation receptors CD5, CD23, CD69, CD80, CD86, and CD25 at various time points (e.g., 6 hrs, 12 hrs, 24 hrs, 48 hrs, etc.) after stimulation using routine art-recognized methods. Suitable antibodies for these receptors are commercially available from, e.g., BD/PharMingen. Effective optimized binuclease fusion proteins are expected to reduce the expression of the lymphocyte activation receptors in stimulated PBMCs relative to controls.

Effects of Optimized Binuclease Fusion Proteins on Plasmacytoid Dendritic Cell (pDC) Interferon Output

pDCs from healthy volunteers are isolated using art-recognized methods or commercially available kits, such as the EasySep™ Human EpCAM Positive Selection Kit (StemCell Technologies, Inc.). Isolated pDCs are cultured in, e.g., 96-well flat-bottom plates, at a densities ranging from 5×10⁴ to 2.5×10⁵/well in 0.1 ml in an appropriate medium (e.g., complete RPMI medium containing 10% FBS, 2 mM glutamine, 55 μM β-mercaptoethanol, 1 mM sodium pyruvate, 100 U/ml penicillin, and 100 μg/ml streptomycin). Cultured pDCs are activated by adding sera or plasma from individual SLE patients diluted with culture medium at a 1:5 ratio, and 0.1 ml of these samples are added to the cell-containing wells (final patient serum concentration is 10%). Cultures are incubated at 37° C. for 40 hr, after which the conditioned media is harvested and assessed for IFNα content using a commercially available ELISA kit. Serum samples obtained from healthy volunteers are used as controls. To assess the impact of the optimized binuclease fusion proteins, SLE patient sera or plasma is pretreated with the optimized binuclease fusion proteins (1-10 μg/ml) for 30 min and added to the pDC cultures. Effective optimized binuclease fusion proteins are expected to reduce the quantity of IFNα produced as a result of degrading the nucleic acid-containing ICs.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

Sequence Table

| SEQ | | |
|---|---|---|
| ID | | |
| NO | Descriptioin | Sequence |
| 1 | RSLV-145 (DNase- | LKIAAFNIQTFGRTKMSNATLVSYIVQILSRYDIALVQEV |
| | NLG linker-RNase- | RDSHLTAVGKLLDNLNQDAPDTYHYVVSEPLGRKSYKERY |
| | Fc) amino acid | LFVYRPDQVSAVDSYYYDDGCEPCGNDTFNREPFIVRFFS |
| | sequence | RFTEVREFAIVPLHAAPGDAVAEIDALYDVYLDVQEKWGL |
| | Mature human DNase1 | EDVMLMGDFNAGCSYVRPSQWSSIRLWTSPTFQWLIPDSA |
| | E13R/N74K/A114F/ | DTTAKPTHCAYDRIVVAGMLLRGAVVPDSALPFNFQAAYG |
| | T205K (underlined) | LSDQLAQAISDHYPVEVMLKVDGASSPVNVSSPSVQDIKE |
| | NLG Linker (bold) | SRAKKFQRQHMDSDSSPSSSSTYCNQMMRRRNMTQGRCKP |
| | Mature human RNase1 | VNTFVHEPLVDVQNVCFQEKVTCKNGQGNCYKSNSSMHIT |
| | (bold underline) | DCRLTNGSRYPNCAYRTSPKERHIIVACEGSPYVPVHFDA |
| | Fc domain | SVEDSTEPKSSDKTHTCPPCPAPELLGGSSVFLFPPKPKD |
| | | TLMISRTPEVTCVVVDVSHEDPEVKFNWYVDGVEVHNAKT |
| | | KPREEQYNSTYRVVSVLTVLHQDWLNGKEYKCKVSNKALP |
| | | ASIEKTISKAKGQPREPQVYTLPPSRDELTKNQVSLTCLV |
| | | KGFYPSDIAVEWESNGQPENNYKTTPPVLDSDGSFFLYSK |
| | | LTVDKSRWQQGNVFSCSVMHEALHNHYTQKSLSLSPGK |
| 2 | RSLV-146 (Fc- | DKTHTCPPCPAPELLGGSSVFLFPPKPKDTLMISRTPEVT |
| | linker-RNase-NLG | CVVVDVSHEDPEVKFNWYVDGVEVHNAKTKPREEQYNSTY |
| | linker-DNase) amino | RVVSVLTVLHQDWLNGKEYKCKVSNKALPASIEKTISKAK |
| | acid sequence | GQPREPQVYTLPPSRDELTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGKGGGGSKESRAKKF |
| | | QRQHMDSDSSPSSSSTYCNQMMRRRNMTQGRCKPVNTFVH |
| | | EPLVDVQNVCFQEKVTCKNGQGNCYKSNSSMHITDCRLTN |
| | | GSRYPNCAYRTSPKERHIIVACEGSPYVPVHFDASVEDST |
| | | VDGASSPVNVSSPSVQDILKIAAFNIQTFGRTKMSNATLV |
| | | SYIVQILSRYDIALVQEVRDSHLTAVGKLLDNLNQDAPDT |
| | | YHYVVSEPLGRKSYKERYLFVYRPDQVSAVDSYYYDDGCE |
| | | PCGNDTFNREPFIVRFFSRFTEVREFAIVPLHAAPGDAVA |
| | | EIDALYDVYLDVQEKWGLEDVMLMGDFNAGCSYVRPSQWS |
| | | SIRLWTSPTFQWLIPDSADTTAKPTHCAYDRIVVAGMLLR |
| | | GAVVPDSALPFNFQAAYGLSDQLAQAISDHYPVEVMLK |
| 3 | RSLV-147 DNase | LKIAAFNIQTFGRTKMSNATLVSYIVQILSRYDIALVQEV |
| | chain (Dnase-FcA) | RDSHLTAVGKLLDNLNQDAPDTYHYVVSEPLGRKSYKERY |
| | amino acid sequence | LFVYRPDQVSAVDSYYYDDGCEPCGNDTFNREPFIVRFFS |
| | | RFTEVREFAIVPLHAAPGDAVAEIDALYDVYLDVQEKWGL |
| | | EDVMLMGDFNAGCSYVRPSQWSSIRLWTSPTFQWLIPDSA |
| | | DTTAKPTHCAYDRIVVAGMLLRGAVVPDSALPFNFQAAYG |
| | | LSDQLAQAISDHYPVEVMLKEPKSSDKTHTCPPCPAPELL |
| | | GGSSVFLFPPKPKDTLMISRTPEVTCVVVDVSHEDPEVKF |
| | | NWYVDGVEVHNAKTKPREEQYNSTYRVVSVLTVLHQDWLN |
| | | GKEYKCKVSNKALPASIEKTISKAKGQPREPQVYVYPPSR |
| | | DELTKNQVSLTCLVKGFYPSDIAVEWESNGQPENNYKTTP |
| | | PVLDSDGSFALVSKLTVDKSRWQQGNVFSCSVMHEALHNH |
| | | YTQKSLSLSPGK |
| 4 | RSLV-147 RNase | KESRAKKFQRQHMDSDSSPSSSSTYCNQMMRRRNMTQGRC |
| | chain (RNase-FcB) | KPVNTFVHEPLVDVQNVCFQEKVTCKNGQGNCYKSNSSMH |
| | amino acid sequence | ITDCRLTNGSRYPNCAYRTSPKERHIIVACEGSPYVPVHF |
| | | DASVEDSTEPKSSDKTHTCPPCPAPELLGGSSVFLFPPKP |
| | | KDTLMISRTPEVTCVVVDVSHEDPEVKFNWYVDGVEVHNA |
| | | KTKPREEQYNSTYRVVSVLTVLHQDWLNGKEYKCKVSNKA |
| | | LPASIEKTISKAKGQPREPQVYVLPPSRDELTKNQVSLLC |
| | | LVKGFYPSDIAVEWESNGQPENNYLTWPPVLDSDGSFFLY |
| | | SKLTVDKSRWQQGNVFSCSVMHEALHNHYTQKSLSLSPGK |
| 5 | RSLV-148 (DNase- | LKIAAFNIQTFGRTKMSNATLVSYIVQILSRYDIALVQEV |
| | FcA-NLG linker- | RDSHLTAVGKLLDNLNQDAPDTYHYVVSEPLGRKSYKERY |
| | RNase) amino acid | LFVYRPDQVSAVDSYYYDDGCEPCGNDTFNREPFIVRFFS |
| | sequence | RFTEVREFAIVPLHAAPGDAVAEIDALYDVYLDVQEKWGL |
| | | EDVMLMGDFNAGCSYVRPSQWSSIRLWTSPTFQWLIPDSA |
| | | DTTAKPTHCAYDRIVVAGMLLRGAVVPDSALPFNFQAAYG |
| | | LSDQLAQAISDHYPVEVMLKEPKSSDKTHTCPPCPAPELL |
| | | GGSSVFLFPPKPKDTLMISRTPEVTCVVVDVSHEDPEVKF |
| | | NWYVDGVEVHNAKTKPREEQYNSTYRVVSVLTVLHQDWLN |
| | | GKEYKCKVSNKALPASIEKTISKAKGQPREPQVYVYPPSR |
| | | DELTKNQVSLTCLVKGFYPSDIAVEWESNGQPENNYKTTP |
| | | PVLDSDGSFALVSKLTVDKSRWQQGNVFSCSVMHEALHNH |
| | | YTQKSLSLSPGKVDGASSPVNVSSPSVQDIKESRAKKFQR |
| | | QHMDSDSSPSSSSTYCNQMMRRRNMTQGRCKPVNTFVHEP |
| | | LVDVQNVCFQEKVTCKNGQGNCYKSNSSMHITDCRLTNGS |
| | | RYPNCAYRTSPKERHIIVACEGSPYVPVHFDASVEDST |
| 6 | RSLV-148 FcB chain | DKTHTCPPCPAPELLGGSSVFLFPPKPKDTLMISRTPEVT |
| | amino acid sequence | CVVVDVSHEDPEVKFNWYVDGVEVHNAKTKPREEQYNSTY |
| | | RVVSVLTVLHQDWLNGKEYKCKVSNKALPASIEKTISKAK |
| | | GQPREPQVYVLPPSRDELTKNQVSLLCLVKGFYPSDIAVE |
| | | WESNGQPENNYLTWPPVLDSDGSFFLYSKLTVDKSRWQQG |
| | | NVFSCSVMHEALHNHYTQKSLSLSPGK |
| 7 | RSLV-149 DNase | LKIAAFNIQTFGRTKMSNATLVSYIVQILSRYDIALVQEV |
| | chain (DNase-Fc) | RDSHLTAVGKLLDNLNQDAPDTYHYVVSEPLGRKSYKERY |
| | amino acid sequence | LFVYRPDQVSAVDSYYYDDGCEPCGNDTFNREPFIVRFFS |
| | | RFTEVREFAIVPLHAAPGDAVAEIDALYDVYLDVQEKWGL |
| | | EDVMLMGDFNAGCSYVRPSQWSSIRLWTSPTFQWLIPDSA |
| | | DTTAKPTHCAYDRIVVAGMLLRGAVVPDSALPFNFQAAYG |
| | | LSDQLAQAISDHYPVEVMLKEPKSSDKTHTCPPCPAPELL |
| | | GGSSVFLFPPKPKDTLMISRTPEVTCVVVDVSHEDPEVKF |
| | | NWYVDGVEVHNAKTKPREEQYNSTYRVVSVLTVLHQDWLN |
| | | GKEYKCKVSNKALPASIEKTISKAKGQPREPQVYVYPPSR |
| | | DELTKNQVSLTCLVKGFYPSDIAVEWESNGQPENNYKTTP |
| | | PVLDSDGSFALVSKLTVDKSRWQQGNVFSCSVMHEALHNH |
| | | YTQKSLSLSPGK |
| 8 | RSLV-149 RNase | DKTHTCPPCPAPELLGGSSVFLFPPKPKDTLMISRTPEVT |
| | chain (Fc-NLG- | CVVVDVSHEDPEVKFNWYVDGVEVHNAKTKPREEQYNSTY |
| | linker-RNase) | RVVSVLTVLHQDWLNGKEYKCKVSNKALPASIEKTISKAK |
| | amino acid sequence | GQPREPQVYVLPPSRDELTKNQVSLLCLVKGFYPSDIAVE |
| | | WESNGQPENNYLTWPPVLDSDGSFFLYSKLTVDKSRWQQG |
| | | NVFSCSVMHEALHNHYTQKSLSLSPGKVDGASSPVNVSSP |
| | | SVQDIKESRAKKFQRQHMDSDSSPSSSSTYCNQMMRRRNM |
| | | TQGRCKPVNTFVHEPLVDVQNVCFQEKVTCKNGQGNCYKS |
| | | NSSMHITDCRLTNGSRYPNCAYRTSPKERHIIVACEGSPY |
| | | VPVHFDASVEDST |
| 9 | RSLV-150 DNase | EPKSCDKTHTCPPCPAPELLGGSSVFLFPPKPKDTLMISR |
| | chain (FcA-NLG | TPEVTCVVVDVSHEDPEVKFNWYVDGVEVHNAKTKPREEQ |
| | linker-DNase) | YNSTYRVVSVLTVLHQDWLNGKEYKCKVSNKALPASIEKT |
| | amino acid sequence | ISKAKGQPREPQVYVYPPSRDELTKNQVSLTCLVKGFYPS |
| | | DIAVEWESNGQPENNYKTTPPVLDSDGSFALVSKLTVDKS |
| | | RWQQGNVFSCSVMHEALHNHYTQKSLSLSPGKVDGASSPV |
| | | NVSSPSVQDILKIAAFNIQTFGRTKMSNATLVSYIVQILS |
| | | RYDIALVQEVRDSHLTAVGKLLDNLNQDAPDTYHYVVSEP |
| | | LGRKSYKERYLFVYRPDQVSAVDSYYYDDGCEPCGNDTFN |
| | | REPFIVRFFSRFTEVREFAIVPLHAAPGDAVAEIDALYDV |
| | | YLDVQEKWGLEDVMLMGDFNAGCSYVRPSQWSSIRLWTSP |
| | | TFQWLIPDSADTTAKPTHCAYDRIVVAGMLLRGAVVPDSA |
| | | LPFNFQAAYGLSDQLAQAISDHYPVEVMLK |
| 10 | RSLV-150 RNase | EPKSCDKTHTCPPCPAPELLGGSSVFLFPPKPKDTLMISR |
| | chain (FcB-NLG | TPEVTCVVVDVSHEDPEVKFNWYVDGVEVHNAKTKPREEQ |
| | linker-RNase) | YNSTYRVVSVLTVLHQDWLNGKEYKCKVSNKALPASIEKT |
| | amino acid sequence | ISKAKGQPREPQVYVLPPSRDELTKNQVSLLCLVKGFYPS |
| | | DIAVEWESNGQPENNYLTWPPVLDSDGSFFLYSKLTVDKS |
| | | RWQQGNVFSCSVMHEALHNHYTQKSLSLSPGKVDGASSPV |
| | | NVSSPSVQDIKESRAKKFQRQHMDSDSSPSSSSTYCNQMM |
| | | RRRNMTQGRCKPVNTFVHEPLVDVQNVCFQEKVTCKNGQG |
| | | NCYKSNSSMHITDCRLTNGSRYPNCAYRTSPKERHIIVAC |
| | | EGSPYVPVHFDASVEDST |
| 11 | RSLV-151 DNase | DKTHTCPPCPAPELLGGSSVFLFPPKPKDTLMISRTPEVT |
| | chain (Fc-NLG | CVVVDVSHEDPEVKFNWYVDGVEVHNAKTKPREEQYNSTY |
| | linker-DNase) | RVVSVLTVLHQDWLNGKEYKCKVSNKALPASIEKTISKAK |
| | amino acid sequence | GQPREPQVYVYPPSRDELTKNQVSLTCLVKGFYPSDIAVE |
| | | WESNGQPENNYKTTPPVLDSDGSFALVSKLTVDKSRWQQG |
| | | NVFSCSVMHEALHNHYTQKSLSLSPGKVDGASSPVNVSSP |
| | | SVQDILKIAAFNIQTFGRTKMSNATLVSYIVQILSRYDIA |
| | | LVQEVRDSHLTAVGKLLDNLNQDAPDTYHYVVSEPLGRKS |
| | | YKERYLFVYRPDQVSAVDSYYYDDGCEPCGNDTFNREPFI |
| | | VRFFSRFTEVREFAIVPLHAAPGDAVAEIDALYDVYLDVQ |
| | | EKWGLEDVMLMGDFNAGCSYVRPSQWSSIRLWTSPTFQWL |
| | | IPDSADTTAKPTHCAYDRIVVAGMLLRGAVVPDSALPFNF |
| | | QAAYGLSDQLAQAISDHYPVEVMLK |
| 12 | RSLV-151 RNase | DKTHTCPPCPAPELLGGSSVFLFPPKPKDTLMISRTPEVT |
| | chain (Fc-NLG | CVVVDVSHEDPEVKFNWYVDGVEVHNAKTKPREEQYNSTY |
| | linker-RNase) | RVVSVLTVLHQDWLNGKEYKCKVSNKALPASIEKTISKAK |
| | amino acid sequence | GQPREPQVYVLPPSRDELTKNQVSLLCLVKGFYPSDIAVE |
| | | WESNGQPENNYLTWPPVLDSDGSFFLYSKLTVDKSRWQQG |
| | | NVFSCSVMHEALHNHYTQKSLSLSPGKVDGASSPVNVSSP |
| | | SVQDIKESRAKKFQRQHMDSDSSPSSSSTYCNQMMRRRNM |
| | | TQGRCKPVNTFVHEPLVDVQNVCFQEKVTCKNGQGNCYKS |
| | | NSSMHITDCRLTNGSRYPNCAYRTSPKERHIIVACEGSPY |
| | | VPVHFDASVEDST |
| 13 | RSLV-152 (RNase- | KESRAKKFQRQHMDSDSSPSSSSTYCNQMMRRRNMTQGRC |
| | Fc-NLG) linker- | KPVNTFVHEPLVDVQNVCFQEKVTCKNGQGNCYKSNSSMH |
| | DNase) amino acid | ITDCRLTNGSRYPNCAYRTSPKERHIIVACEGSPYVPVHF |
| | sequence | DASVEDSTEPKSSDKTHTCPPCPAPELLGGSSVFLFPPKP |
| | | KDTLMISRTPEVTCVVVDVSHEDPEVKFNWYVDGVEVHNA |
| | | KTKPREEQYNSTYRVVSVLTVLHQDWLNGKEYKCKVSNKA |
| | | LPASIEKTISKAKGQPREPQVYVYPPSRDELTKNQVSLTC |
| | | LVKGFYPSDIAVEWESNGQPENNYKTTPPVLDSDGSFALV |
| | | SKLTVDKSRWQQGNVFSCSVMHEALHNHYTQKSLSLSPGK |
| | | VDGASSPVNVSSPSVQDILKIAAFNIQTFGRTKMSNATLV |
| | | SYIVQILSRYDIALVQEVRDSHLTAVGKLLDNLNQDAPDT |
| | | YHYVVSEPLGRKSYKERYLFVYRPDQVSAVDSYYYDDGCE |
| | | PCGNDTFNREPFIVRFFSRFTEVREFAIVPLHAAPGDAVA |
| | | EIDALYDVYLDVQEKWGLEDVMLMGDFNAGCSYVRPSQWS |
| | | SIRLWTSPTFQWLIPDSADTTAKPTHCAYDRIVVAGMLLR |
| | | GAVVPDSALPFNFQAAYGLSDQLAQAISDHYPVEVMLK |
| 14 | RSLV-152 Fc | DKTHTCPPCPAPELLGGSSVFLFPPKPKDTLMISRTPEVT |
| | domain chain amino | CVVVDVSHEDPEVKFNWYVDGVEVHNAKTKPREEQYNSTY |
| | acid sequence | RVVSVLTVLHQDWLNGKEYKCKVSNKALPASIEKTISKAK |
| | | GQPREPQVYVLPPSRDELTKNQVSLLCLVKGFYPSDIAVE |
| | | WESNGQPENNYLTWPPVLDSDGSFFLYSKLTVDKSRWQQG |
| | | NVFSCSVMHEALHNHYTQKSLSLSPGK |
| 15 | RSLV-153 DNase | DKTHTCPPCPAPELLGGSSVFLFPPKPKDTLMISRTPEVT |
| | chain (Fc-NLG | CVVVDVSHEDPEVKFNWYVDGVEVHNAKTKPREEQYNSTY |
| | linker-DNase) | RVVSVLTVLHQDWLNGKEYKCKVSNKALPASIEKTISKAK |
| | amino acid sequence | GQPREPQVYVYPPSRDELTKNQVSLTCLVKGFYPSDIAVE |
| | | WESNGQPENNYKTTPPVLDSDGSFALVSKLTVDKSRWQQG |
| | | NVFSCSVMHEALHNHYTQKSLSLSPGKVDGASSPVNVSSP |
| | | SVQDILKIAAFNIQTFGRTKMSNATLVSYIVQILSRYDIA |
| | | LVQEVRDSHLTAVGKLLDNLNQDAPDTYHYVVSEPLGRKS |
| | | YKERYLFVYRPDQVSAVDSYYYDDGCEPCGNDTFNREPFI |
| | | VRFFSRFTEVREFAIVPLHAAPGDAVAEIDALYDVYLDVQ |
| | | EKWGLEDVMLMGDFNAGCSYVRPSQWSSIRLWTSPTFQWL |
| | | IPDSADTTAKPTHCAYDRIVVAGMLLRGAVVPDSALPFNF |
| | | QAAYGLSDQLAQAISDHYPVEVMLK |
| 16 | RSLV-153 RNase | KESRAKKFQRQHMDSDSSPSSSSTYCNQMMRRRNMTQGRC |
| | chain (RNase-Fc) | KPVNTFVHEPLVDVQNVCFQEKVTCKNGQGNCYKSNSSMH |
| | amino acid sequence | ITDCRLTNGSRYPNCAYRTSPKERHIIVACEGSPYVPVHF |
| | | DASVEDSTEPKSSDKTHTCPPCPAPELLGGSSVFLFPPKP |
| | | KDTLMISRTPEVTCVVVDVSHEDPEVKFNWYVDGVEVHNA |
| | | KTKPREEQYNSTYRVVSVLTVLHQDWLNGKEYKCKVSNKA |
| | | LPASIEKTISKAKGQPREPQVYVLPPSRDELTKNQVSLLC |
| | | LVKGFYPSDIAVEWESNGQPENNYLTWPPVLDSDGSFFLY |
| | | SKLTVDKSRWQQGNVFSCSVMHEALHNHYTQKSLSLSPGK |
| 17 | RSLV-154 (RNase- | KESRAKKFQRQHMDSDSSPSSSSTYCNQMMRRRNMTQGRC |
| | Fc-NLG linker- | KPVNTFVHEPLVDVQNVCFQEKVTCKNGQGNCYKSNSSMH |
| | DNase) amino acid | ITDCRLTNGSRYPNCAYRTSPKERHIIVACEGSPYVPVHF |
| | sequence | DASVEDSTELLGGSSVFLFPPKPKDTLMISRTPEVTCVVV |
| | | DVSHEDPEVKFNWYVDGVEVHNAKTKPREEQYNSTYRVVS |
| | | VLTVLHQDWLNGKEYKCKVSNKALPASIEKTISKAKGQPR |
| | | EPQVYTLPPSRDELTKNQVNLTCLVKGFYPSDIAVEWESN |
| | | GQPENNYKTTPPVLDSDGSFFLNSTLTVDKSRWQQGNVFS |
| | | CSVMHEALHNHYTQKSLSLSPGKVDGASSPVNVSSPSVQD |
| | | ILKIAAFNIQTFGRTKMSNATLVSYIVQILSRYDIALVQE |
| | | VRDSHLTAVGKLLDNLNQDAPDTYHYVVSEPLGRKSYKER |
| | | YLFVYRPDQVSAVDSYYYDDGCEPCGNDTFNREPFIVRFF |
| | | SRFTEVREFAIVPLHAAPGDAVAEIDALYDVYLDVQEKWG |
| | | LEDVMLMGDFNAGCSYVRPSQWSSIRLWTSPTFQWLIPDS |
| | | ADTTAKPTHCAYDRIVVAGMLLRGAVVPDSALPFNFQAAY |
| | | GLSDQLAQAISDHYPVEVMLK |
| 18 | Fc-NLG-linker-DNAse | DKTHTCPPCPAPELLGGSSVFLFPPKPKDTLMISRTPEVT |
| | (control construct) | CVVVDVSHEDPEVKFNWYVDGVEVHNAKTKPREEQYNSTY |
| | | RVVSVLTVLHQDWLNGKEYKCKVSNKALPASIEKTISKAK |
| | | GQPREPQVYTLPPSRDELTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGKVDGASSPVNVSSP |
| | | SVQDILKIAAFNIQTFGETKMSNATLVSYIVQILSRYDIA |
| | | LVQEVRDSHLTAVGKLLDNLNQDAPDTYHYVVSEPLGRNS |
| | | YKERYLFVYRPDQVSAVDSYYYDDGCEPCRNDTFNREPFI |
| | | VRFFSRFTEVREFAIVPLHAAPGDAVAEIDALYDVYLDVQ |
| | | EKWGLEDVMLMGDFNAGCSYVRPSQWSSIRLWTSPTFQWL |
| | | IPDSADTTATPTHCAYDRIVVAGMLLRGAVVPDSALPFNF |
| | | QAAYGLSDQLAQAISDHYPVEVMLK |
| 19 | DNASe-Fc | LKIAAFNIQTFGRTKMSNATLVSYIVQILSRYDIALVQEV |
| | (control construct) | RDSHLTAVGKLLDNLNQDAPDTYHYVVSEPLGRKSYKERY |
| | | LFVYRPDQVSAVDSYYYDDGCEPCGNDTFNREPFIVRFFS |
| | | RFTEVREFAIVPLHAAPGDAVAEIDALYDVYLDVQEKWGL |
| | | EDVMLMGDFNAGCSYVRPSQWSSIRLWTSPTFQWLIPDSA |
| | | DTTAKPTHCAYDRIVVAGMLLRGAVVPDSALPFNFQAAYG |
| | | LSDQLAQAISDHYPVEVMLKEPKSSDKTHTCPPCPAPELL |
| | | GGSSVFLFPPKPKDTLMISRTPEVTCVVVDVSHEDPEVKF |
| | | NWYVDGVEVHNAKTKPREEQYNSTYRVVSVLTVLHQDWLN |
| | | GKEYKCKVSNKALPASIEKTISKAKGQPREPQVYTLPPSR |
| | | DELTKNQVSLTCLVKGFYPSDIAVEWESNGQPENNYKTTP |
| | | PVLDSDGSFFLYSKLTVDKSRWQQGNVFSCSVMHEALHNH |
| | | YTQKSLSLSPGK |
| 20 | Mature wild type | LKIAAFNIQTFGETKMSNATLVSYIVQILSRYDIALVQEV |
| | Human DNase1 | RDSHLTAVGKLLDNLNQDAPDTYHYVVSEPLGRNSYKERY |
| | | LFVYRPDQVSAVDSYYYDDGCEPCGNDTFNREPAIVRFFS |
| | | RFTEVREFAIVPLHAAPGDAVAEIDALYDVYLDVQEKWGL |
| | | EDVMLMGDFNAGCSYVRPSQWSSIRLWTSPTFQWLIPDSA |
| | | DTTATPTHCAYDRIVVAGMLLRGAVVPDSALPFNFQAAYG |
| | | LSDQLAQAISDHYPVEVMLK |
| 21 | Mature human | LKIAAFNIQTFGETKMSNATLVSYIVQILSRYDIALVQEV |
| | DNase1 A114F | RDSHLTAVGKLLDNLNQDAPDTYHYVVSEPLGRNSYKERY |
| | | LFVYRPDQVSAVDSYYYDDGCEPCGNDTFNREPFIVRFFS |
| | | RFTEVREFAIVPLHAAPGDAVAEIDALYDVYLDVQEKWGL |
| | | EDVMLMGDFNAGCSYVRPSQWSSIRLWTSPTFQWLIPDSA |
| | | DTTATPTHCAYDRIVVAGMLLRGAVVPDSALPFNFQAAYG |
| | | LSDQLAQAISDHYPVEVMLK |
| 22 | Mature human | LKIAAFNIQTFGETKMSNATLVSYIVQILSRYDIALVQEV |
| | DNase1 G105R | RDSHLTAVGKLLDNLNQDAPDTYHYVVSEPLGRNSYKERY |
| | | LFVYRPDQVSAVDSYYYDDGCEPCRNDTFNREPAIVRFFS |
| | | RFTEVREFAIVPLHAAPGDAVAEIDALYDVYLDVQEKWGL |
| | | EDVMLMGDFNAGCSYVRPSQWSSIRLWTSPTFQWLIPDSA |
| | | DTTATPTHCAYDRIVVAGMLLRGAVVPDSALPFNFQAAYG |
| | | LSDQLAQAISDHYPVEVMLK |
| 23 | Precursor human | MRGMKLLGALLALAALLQGAVSLKIAAFNIQTFGETKMSN |
| | DNase1 | ATLVSYIVQILSRYDIALVQEVRDSHLTAVGKLLDNLNQD |
| | | APDTYHYVVSEPLGRNSYKERYLFVYRPDQVSAVDSYYYD |
| | | DGCEPCGNDTFNREPAIVRFFSRFTEVREFAIVPLHAAPG |
| | | DAVAEIDALYDVYLDVQEKWGLEDVMLMGDFNAGCSYVRP |
| | | SQWSSIRLWTSPTFQWLIPDSADTTATPTHCAYDRIVVAG |
| | | MLLRGAVVPDSALPFNFQAAYGLSDQLAQAISDHYPVEV |
| | | MLK |
| 24 | Mature human DNase1 | LKIAAFNIQTFGETKMSSATLVSYIVQILSRYDIALVQEV |
| | N18S/N106S/A114F | RDSHLTAVGKLLDNLNQDAPDTYHYVVSEPLGRNSYKERY |
| | | LFVYRPDQVSAVDSYYYDDGCEPCGSDTFNREPFIVRFFS |
| | | RFTEVREFAIVPLHAAPGDAVAEIDALYDVYLDVQEKWGL |
| | | EDVMLMGDFNAGCSYVRPSQWSSIRLWTSPTFQWLIPDSA |
| | | DTTATPTHCAYDRIVVAGMLLRGAVVPDSALPFNFQAAYG |
| | | LSDQLAQAISDHYPVEVMLK |
| 25 | Mature human DNase1 | LKIAAFNIQTFGRTKMSNATLVSYIVQILSRYDIALVQEV |
| | E13R/N74K/A114F/ | RDSHLTAVGKLLDNLNQDAPDTYHYVVSEPLGRKSYKERY |
| | T205K | LFVYRPDQVSAVDSYYYDDGCEPCGNDTFNREPFIVRFFS |
| | | RFTEVREFAIVPLHAAPGDAVAEIDALYDVYLDVQEKWGL |
| | | EDVMLMGDFNAGCSYVRPSQWSSIRLWTSPTFQWLIPDSA |
| | | DTTAKPTHCAYDRIVVAGMLLRGAVVPDSALPFNFQAAYG |
| | | LSDQLAQAISDHYPVEVMLK |
| 26 | Mature human DNase1 | LKIAAFNIQTFGRTKMSSATLVSYIVQILSRYDIALVQEV |
| | E13R/N74K/A114F/ | RDSHLTAVGKLLDNLNQDAPDTYHYVVSEPLGRKSYKERY |
| | T205K/N18S/N106S | LFVYRPDQVSAVDSYYYDDGCEPCGSDTFNREPFIVRFFS |
| | | RFTEVREFAIVPLHAAPGDAVAEIDALYDVYLDVQEKWGL |
| | | EDVMLMGDFNAGCSYVRPSQWSSIRLWTSPTFQWLIPDSA |
| | | DTTAKPTHCAYDRIVVAGMLLRGAVVPDSALPFNFQAAYG |
| | | LSDQLAQAISDHYPVEVMLK |
| 27 | Mature human RNase1 | KESRAKKFQRQHMDSDSSPSSSSTYCNQMMRRRNMTQGRC |
| | | KPVNTFVHEPLVDVQNVCFQEKVTCKNGQGNCYKSNSSMH |
| | | ITDCRLTNGSRYPNCAYRTSPKERHIIVACEGSPYVPVHF |
| | | DASVEDST |
| 28 | Mature human RNase1 | KESRAKKFQRQHMDSDSSPSSSSTYCNQMMRRRSMTQGRC |
| | N34S/N76S/N88S | KPVNTFVHEPLVDVQNVCFQEKVTCKNGQGNCYKSSSSMH |
| | | ITDCRLTSGSRYPNCAYRTSPKERHIIVACEGSPYVPVHF |
| | | DASVEDST |
| 29 | Precursor human | MALEKSLVRLLLLVLILLVLGWVQPSLGKESRAKKFQRQH |
| | RNase1 | MDSDSSPSSSSTYCNQMMRRRNMTQGRCKPVNTFVHEPLV |
| | | DVQNVCFQEKVTCKNGQGNCYKSNSSMHITDCRLTNGSRY |
| | | PNCAYRTSPKERHIIVACEGSPYVPVHFDASVEDST |
| 30 | (Gly₄Ser)3 linker | GGGGSGGGGSGGGGS |
| 31 | Gaussia luciferase | MGVKVLFALICIAVAEA |
| | signal peptide | |
| 32 | Linker | LEA(EAAAK)₄ALEA(EAAAK)₄ |
| 33 | O-linked | CXXGG-T/S-C |
| | glycosylation | |
| | consensus | |
| 34 | O-linked | NST-E/D-A |
| | glycosylation | |
| | consensus | |
| 35 | O-linked | NITQS |
| | glycosylation | |
| | consensus | |
| 36 | O-linked | QSTQS |
| | glycosylation | |
| | consensus | |
| 37 | O-linked | D/EFT-R/K-V |
| | glycosylation | |
| | consensus | |
| 38 | O-linked | C-E/D-SN |
| | glycosylation | |
| | consensus | |
| 39 | O-linked | GGSC-K/R |
| | glycosylation | |
| | consensus | |
| 40 | VK3 light chain | METPAQLLFLLLLWLPDTTG |
| | signal peptide | |
| 41 | NLG linker | VDGASSPVNVSSPSVQDI |
| 42 | linker | LEA(EAAAK)₄ALEA(EAAAK)₄ALE |
| 43 | Linker | GGSG |
| 44 | Linker | GSAT |
| 45 | Human wild-type | EPKSCDKTHTCPPCPAPELLGGPSVFLFPPKPKDTLMISR |
| | IgG1 Fc domain | TPEVTCVVVDVSHEDPEVKFNWYVDGVEVHNAKTKPREEQ |
| | | YNSTYRVVSVLTVLHQDWLNGKEYKCKVSNKALPAPIEKT |
| | | ISKAKGQPREPQVYTLPPSRDELTKNQVSLTCLVKGFYPS |
| | | DIAVEWESNGQPENNYKTTPPVLDSDGSFFLYSKLTVDKS |
| | | RWQQGNVFSCSVMHEALHNHYTQKSLSLSPGK |
| 46 | Mature human | MRICSFNVRSFGESKQEDKNAMDVIVKVIKRCDIILVMEI |
| | DNase1L3 | KDSNNRICPILMEKLNRNSRRGITYNYVISSRLGRNTYKE |
| | | QYAFLYKEKLVSVKRSYHYHDYQDGDADVFSREPFVVWFQ |
| | | SPHTAVKDFVIIPLHTTPETSVKEIDELVEVYTDVKHRWK |
| | | AENFIFMGDFNAGCSYVPKKAWKNIRLRTDPRFVWLIGDQ |
| | | EDTTVKKSTNCAYDRIVLRGQEIVSSVVPKSNSVFDFQKA |
| | | YKLTEEEALDVSDHFPVEFKLQSSRAFTNSKKSVTLRKKT |
| | | KSKRS |
| 47 | Human Trex1 | MGPGARRQGRIVQGRPEMCFCPPPTPLPPLRILTLGTHTP |
| | | TPCSSPGSAAGTYPTMGSQALPPGPMQTLIFFDMEATGLP |
| | | FSQPKVTELCLLAVHRCALESPPTSQGPPPTVPPPPRVVD |
| | | KLSLCVAPGKACSPAASEITGLSTAVLAAHGRQCFDDNLA |
| | | NLLLAFLRRQPQPWCLVAHNGDRYDFPLLQAELAMLGLTS |
| | | ALDGAFCVDSITALKALERASSPSEHGPRKSYSLGSIYTR |
| | | LYGQSPPDSHTAEGDVLALLSICQWRPQALLRWVDAHARP |
| | | FGTIRPMYGVTASARTKPRPSAVTTTAHLATTRNTSPSLG |
| | | ESRGTKDLPPVKDPGALSREGLLAPLGLLAILTLAVATLY |
| | | GLSLATPGE |
| 48 | Human DNase2 alpha | MIPLLLAALLCVPAGALTCYGDSGQPVDWFVVYKLPALRG |
| | (NP_001366.1) | SGEAAQRGLQYKYLDESSGGWRDGRALINSPEGAVGRSLQ |
| | | PLYRSNTSQLAFLLYNDQPPQPSKAQDSSMRGHTKGVLLL |
| | | DHDGGFWLVHSVPNFPPPASSAAYSWPHSACTYGQTLLCV |
| | | SFPFAQFSKMGKQLTYTYPWVYNYQLEGIFAQEFPDLENV |
| | | VKGHHVSQEPWNSSITLTSQAGAVFQSFAKFSKFGDDLYS |
| | | GWLAAALGTNLQVQFWHKTVGILPSNCSDIWQVLNVNQIA |
| | | FPGPAGPSFNSTEDHSKWCVSPKGPWTCVGDMNRNQGEEQ |
| | | RGGGTLCAQLPALWKAFQPLVKNYQPCNGMARKPSRAYKI |
| 49 | human DNase2 beta | MKQKMMARLLRTSFALLFLGLFGVLGAATISCRNEEGKAV |
| | | DWFTFYKLPKRQNKESGETGLEYLYLDSTTRSWRKSEQLM |
| | | NDTKSVLGRTLQQLYEAYASKSNNTAYLIYNDGVPKPVNY |
| | | SRKYGHTKGLLLWNRVQGFWLIHSIPQFPPIPEEGYDYPP |
| | | TGRRNGQSGICITFKYNQYEAIDSQLLVCNPNVYSCSIPA |
| | | TFHQELIHMPQLCTRASSSEIPGRLLTTLQSAQGQKFLHF |
| | | AKSDSFLDDIFAAWMAQRLKTHLLTETWQRKRQELPSNCS |
| | | LPYHVYNIKAIKLSRHSYFSSYQDHAKWCISQKGTKNRWT |
| | | CIGDLNRSPHQAFRSGGFICTQNWQIYQAFQGLVLYYESC |
| | | K |
| 50 | Fc region N83S | EPKSCDKTHTCPPCPAPELLGGPSVFLFPPKPKDTLMISR |
| | | TPEVTCVVVDVSHEDPEVKFNWYVDGVEVHNAKTKPREEQ |
| | | YSSTYRVVSVLTVLHQDWLNGKEYKCKVSNKALPAPIEKT |
| | | ISKAKGQPREPQVYTLPPSRDELTKNQVSLTCLVKGFYPS |
| | | DIAVEWESNGQPENNYKTTPPVLDSDGSFFLYSKLTVDKS |
| | | RWQQGNVFSCSVMHEALHNHYTQKSLSLSPGK |
| 51 | Fc region with SCC | EPKSSDKTHTCPPCPAPELLGGPSVFLFPPKPKDTLMISR |
| | | TPEVTCVVVDVSHEDPEVKFNWYVDGVEVHNAKTKPREEQ |
| | | YNSTYRVVSVLTVLHQDWLNGKEYKCKVSNKALPAPIEKT |
| | | ISKAKGQPREPQVYTLPPSRDELTKNQVSLTCLVKGFYPS |
| | | DIAVEWESNGQPENNYKTTPPVLDSDGSFFLYSKLTVDKS |
| | | RWQQGNVFSCSVMHEALHNHYTQKSLSLSPGK |
| 52 | Fc regi on with SSS | EPKSSDKTHTSPPSPAPELLGGPSVFLFPPKPKDTLMISR |
| | | TPEVTCVVVDVSHEDPEVKFNWYVDGVEVHNAKTKPREEQ |
| | | YNSTYRVVSVLTVLHQDWLNGKEYKCKVSNKALPAPIEKT |
| | | ISKAKGQPREPQVYTLPPSRDELTKNQVSLTCLVKGFYPS |
| | | DIAVEWESNGQPENNYKTTPPVLDSDGSFFLYSKLTVDKS |
| | | RWQQGNVFSCSVMHEALHNHYTQKSLSLSPGK |
| 53 | Linker | LEA(EAAAK)₄ALEA(EAAAK)₄ALE |
| 54 | VK3LP leader | METPAQLLFLLLLWLPDTTG |
| 55 | Fc region with SCC, | EPKSSDKTHTSPPSPAPELLGGSSVFLFPPKPKDTLMISR |
| | P238S, P331S | TPEVTCVVVDVSHEDPEVKFNWYVDGVEVHNAKTKPREEQ |
| | mutations | YNSTYRVVSVLTVLHQDWLNGKEYKCKVSNKALPASIEKT |
| | | ISKAKGQPREPQVYTLPPSRDELTKNQVSLTCLVKGFYPS |
| | | DIAVEWESNGQPENNYKTTPPVLDSDGSFFLYSKLTVDKS |
| | | RWQQGNVFSCSVMHEALHNHYTQKSLSLSPGK |
| 56 | Fc region with | EPKCSDKTHTSPPSPAPELLGGSSVFLFPPKPKDTLMISR |
| | P238S, P331S | TPEVTCVVVDVSHEDPEVKFNWYVDGVEVHNAKTKPREEQ |
| | mutations | YNSTYRVVSVLTVLHQDWLNGKEYKCKVSNKALPASIEKT |
| | | ISKAKGQPREPQVYTLPPSRDELTKNQVSLTCLVKGFYPS |
| | | DIAVEWESNGQPENNYKTTPPVLDSDGSFFLYSKLTVDKS |
| | | RWQQGNVFSCSVMHEALHNHYTQKSLSLSPGK |
| 57 | RSLV-327 | METPAQLLFLLLLWLPDTTGKESRAKKFQRQHMDSDSSPS |
| | (RNase-linker-HSA- | SSSTYCNQMMRRRNMTQGRCKPVNTFVHEPLVDVQNVCFQ |
| | linker-DNase | EKVTCKNGQGNCYKSNSSMHITDCRLTNGSRYPNCAYRTS |
| | E13R/N74K/A114F/ | PKERHIIVACEGSPYVPVHFDASVEDSTGGGGSGGGGSGG |
| | T205K) | GGSDAHKSEVAHRFKDLGEENFKALVLIAFAQYLQQCPFE |
| | | DHVKLVNEVTEFAKTCVADESAENCDKSLHTLFGDKLCTV |
| | | ATLRETYGEMADCCAKQEPERNECFLQHKDDNPNLPRLVR |
| | | PEVDVMCTAFHDNEETFLKKYLYEIARRHPYFYAPELLFF |
| | | AKRYKAAFTECCQAADKAACLLPKLDELRDEGKASSAKQR |
| | | LKCASLQKFGERAFKAWAVARLSQRFPKAEFAEVSKLVTD |
| | | LTKVHTECCHGDLLECADDRADLAKYICENQDSISSKLKE |
| | | CCEKPLLEKSHCIAEVENDEMPADLPSLAADFVESKDVCK |
| | | NYAEAKDVFLGMFLYEYARRHPDYSVVLLLRLAKTYETTL |
| | | EKCCAAADPHECYAKVFDEFKPLVEEPQNLIKQNCELFEQ |
| | | LGEYKFQNALLVRYTKKVPQVSTPTLVEVSRNLGKVGSKC |
| | | CKHPEAKRMPCAEDYLSVVLNQLCVLHEKTPVSDRVTKCC |
| | | TESLVNRRPCFSALEVDETYVPKEFNAETFTFHADICTLS |
| | | EKERQIKKQTALVELVKHKPKATKEQLKAVMDDFAAFVEK |
| | | CCKADDKETCFAEEGKKLVAASQAALGLGGGGSGGGGSGG |
| | | GGSLKIAAFNIQTFGRTKMSNATLVSYIVQILSRYDIALV |
| | | QEVRDSHLTAVGKLLDNLNQDAPDTYHYVVSEPLGRKSYK |
| | | ERYLFVYRPDQVSAVDSYYYDDGCEPCGNDTFNREPFIVR |
| | | FFSRFTEVREFAIVPLHAAPGDAVAEIDALYDVYLDVQEK |
| | | WGLEDVMLMGDFNAGCSYVRPSQWSSIRLWTSPTFQWLIP |
| | | DSADTTAKPTHCAYDRIVVAGMLLRGAVVPDSALPFNFQA |
| | | AYGLSDQLAQAISDHYPVEVMLK |

1.-5. (canceled)

6. A method for degrading immune complexes containing RNA, DNA, or both RNA and DNA, comprising administering to a subject a composition comprising a homodimer, wherein the homodimer comprises a polypeptide comprising a first nuclease domain, a second nuclease domain, and an Fc domain, wherein the first nuclease domain is human DNase1 and the second nuclease domain is human RNase1, wherein the human DNase1 is operably linked with or without a linker in tandem from N- to C-terminus to the human RNase1, wherein the human RNase1 is operably linked with or without a linker to the Fc domain, and wherein the polypeptide comprises an amino acid sequence at least 90% identical to the amino acid sequence of SEQ ID NO:1.

7. The method of claim 6, wherein the polypeptide comprises an amino acid sequence at least 95% identical to the amino acid sequence of SEQ ID NO: 1.

8. The method of claim 6, wherein the polypeptide comprises an amino acid sequence is at least 97% identical to the amino acid sequence of SEQ ID NO: 1.

9. The method of claim 6, wherein the polypeptide comprises the amino acid sequence of SEQ ID NO: 1.

10. The method of claim 6, wherein the composition further comprises a pharmaceutically acceptable carrier.

11. The method of claim 6, wherein the method comprises degrading immune complexes in vitro.

12. The method of claim 6, wherein the method comprises degrading immune complexes in vivo.

13. The method of claim 7, wherein the composition further comprises a pharmaceutically acceptable carrier.

14. The method of claim 7, wherein the method comprises degrading immune complexes in vitro.

15. The method of claim 7, wherein the method comprises degrading immune complexes in vivo.

16. The method of claim 8, wherein the composition further comprises a pharmaceutically acceptable carrier.

17. The method of claim 8, wherein the method comprises degrading immune complexes in vitro.

18. The method of claim 8, wherein the method comprises degrading immune complexes in vivo.

19. The method of claim 9, wherein the composition further comprises a pharmaceutically acceptable carrier.

20. The method of claim 9, wherein the method comprises degrading immune complexes in vitro.

21. The method of claim 9, wherein the method comprises degrading immune complexes in vivo.
